SEQUENCE LISTING (76 sequences)

SEQ ID NO: 1 - DNA, 732 bp, Zea mays
atggcgagcgccgatctgctgcggaaggaggaggagttctactcctccctctttgattccgcaaaaggcgacggcgtcaagtcgcgctcgcaggtgattgagaggaagattgaatccctcgaggacatggccaccaaggtcagcaaccggagatcaagaagatggttgaacgaccgcttgctgattgagcttgtcccacgccttcatgttgaagaaatcaaaggcctctttgctcctccaccatggggtgaggagctgcccttgtcagcattctgcaggacaagtgttagtgactgggaggccttcaggagcatcgacatggatgctgaggcaagattgatgcaacacatgaaacgatcatccgaaaaacaaaggactcatgtcgacgaagatgaattgattgctctgaatgcttggcgtcgtataggtcgccaaacaagagaagctattaagaaaaaatttctacctgatctacttcaaatatatgaagaacaggttagggccttcattgaaggtactggtgacagcgacgtgcttgtgctgaatgtccaggacccgttccagaggctgcttctgcacggtgtttgtgagttctataacgtaacctcaatgaccacaagcagtgtaagggacgggaagccatggaagagcaccaccatcaagaagaggcatgtcaccggtcttcctccgagaatcacattagttagcttcctgaggatgaggaagaattag

SEQ ID NO: 2 - DNA, 987 bp, Synechocystis sp.
atgtctgaaaattttgcagttgctacgccggtgcgggtcggaattgtcggtactggttatgcggcccaacgtcgggcggaagttttccggggcgatcgccgtagtcaattggttagtttttggggcaatagtgaagccaatacagctaaatttgccgatacttttggagttagaccccagcaatcttggcaggcattaattaatgatccagagatagatttagtgctcattgccaccattaaccaactccatggggcgatcgccgaggcggcattgcaagccggtaaacatgtggtgttggaatatcctttagcgttaacctatgccatgggcaaaaaactacaacagttagcccgggaaaaaggtaaattactgcatgtggaacatattgaactattggggggagtacaccaagccattcgccagaacctaggcaaaattggtgaggttttttacgcccgctatagcaccatcatgggacaaaatcccgctccccaacgttggacctatcaccatcagcaatttggctttcctttagtggcggccttgtcccgcatcagtcggtttacggatttattcggtacagtacagcaggtggatgcccaatgtcgtttttgggatcagcctaatccggaatattttcgggcttgtttagccaccgcctatctccagtttaataatggtcttaaagcggaggttatctatggcaaaggggaagtttttcaccagaatgaacggatttttaccctccatggcgatcgaggcaccttaatttttgtcggggaaacaggtaggttaattcagggacaaacggaaactgaaattaccgttggtagtcgtcgaggactgttcagacaagacacggaagcagtgttggattatctaaccactggtaagcccctttatgtggatttagaagctagtttatatgctttagaagtggcggatctctgtgcccaagcttgtggatataaggttgaaaattag

SEQ ID NO: 3 - DNA, 987 bp, Zea mays; identical to SEQ ID NO: 2

SEQ ID NO: 4 - DNA, 987 bp, Zea mays; identical to SEQ ID NO: 2

SEQ ID NO: 5 - DNA, 786 bp, Glycine max
atgactgcctcaaaggatcgcgagaacttcgtctacatcgccaaactcgccgagcaggccgagcgttacgaagagatggtggaatcaatgaagaacgttgcgaatctcgacgttgaactgacggtggaggagcggaatttgctttctgttgggtacaagaatgtgattggtgctcgcagagcgtcctggaggatcctgtcttccattgagcagaaggaagaaacaaaagggaacgagttgaatgcgaagcggattaaggagtacagacagaaggttgaattggagctatcgaacatctgtaatgatgttatgagagtgattgatgagcaccttatcccttcggctgcagctggtgaatccactgtgttttactataagatgaaaggagattattatcgttatcttgctgaatttaagtcaggcaatgagaagaaggaagctgctgatcagtctatgaaagcatatgagtctgctaccgctgcagcagaggctgatttacctcccacacatcccatccgattgggtctagctttaaatttctctgttttctattatgagatcttgaactcccctgaaagagcctgtcatcttgcgaagcaagcttttgatgaagctatttctgagcttgacaccttgaatgaggagtcatacaaagatagcaccttaatcatgcaacttctcagggacaacctaactttgtggacttctgacatcccagaagatggagaagatgcccagaaagtgaatggcactgccaagcttggtggaggtgaggatgcagagtag

SEQ ID NO: 6 - DNA, 1041 bp, Synechocystis sp.
atgaccgttagtgagattcatattcctaactctttactagaccgggattgcaccaccctttcacgccacgtactccaacaactgaatagctttggggccgatgcccaggatttgagtgccatcatgaaccgcattgccctagcgggaaaactgattgcccgtcgcctgagtcgagctgggttaatggccgatgtgttgggcttcactggggaaaccaacgtccagggggaatcggtgaaaaaaatggacgtatttgccaatgatgtttttatttctgtctttaagcaaagtggcttggtttgtcgtctggcttcggaggagatggaaaaaccctactatattcctgaaaattgccccattggtcgctatactttgctgtacgaccccattgatggttcctccaacgtggacattaacctcaacgtgggttccatttttgccattcggcaacaggaaggggacgatctagacggcagtgcgtcagatttattggctaacggagacaagcaaattgctgctggttatatcctctacggcccctccaccatcctggtttattccctcggctccggagtgcatagctttatcctcgatcccagtttgggggaatttattttagcccaggaaaatatccgcattcccaaccacggccccatttacagcaccaatgaaggtaacttttggcaatgggatgaagccctgagggattacacccgttacgtccatcgccacgaaggttacactgcccgttatagcggtgctctggtgggggatattcaccggattttgatgcaagggggagtgtttctttatcctggtacggaaaaaaatcccgacggcaaattgcgtttgctctatgaaactgcgccgctggcctttttggtggaacaggctgggggaagggctagtgacggccaaaaacgtttactggacttaattccttctaaattacatcagcgtacccccgccattattggcagcgcagaagatgtgaaattggtggaatctttcatcagcgaccacaaacaacggcagggtaat

SEQ ID NO: 7 - DNA, 1473 bp, Zea mays
atggctccatctccgcctcggcgtgctgcgctcctcctcaccactgtgcttctgctggcggcggccgggtccacccccggcgccacggctgctggcatcttccaggtgcgccgcaagttcaccgccggggtgggagggggtgctggcgccaacatcagcgcccttcgcgcccacgacggcacccgtcacggccgcctcctcgcagccgccgacctccctcttggcggcctcggcctccccactgacactggcctctattacacggagatcaagctcgggacgccacccaagcactactacgtccaggtcgacaccggcagcgacatcctctgggtcaactgcatcacctgcgagcaatgcccccacaagagcgggctcgggttagacttgacgctttacgaccccaaggcatcctcgaccgggagcatggtgatgtgcgatcaggcattctgtgcagccacctttggcggaaagctgccgaagtgcggcgccaatgtgccctgcgaatatagtgtcacctacggtgatggtagctcgacgataggttcctttgtcaccgatgcgctgcagtttgatcaggtgactagggatgggcagacacagccggctaatgccagcgtcattttcgggtgtggtgctcaacaaggtggggatttgggaagttcaaaccaagcccttgatggaattcttggttttggtgaggcaaatacatcaatgctatcacaactaactaccgctggtaaagtgaagaagatatttgcccattgtttggataccataaaaggaggtggaattttttctattggagatgtggtgcagccaaaagtaaaaacaacgccattggtagccgacaagccacactacaatgtgaaccttaagacaatcgatgttggtggcactactctacaactcccagctcatatttttgaaccaggagaaaaaaagggtaccataatcgacagtggtaccaccttgacataccttccagaattggttttcaaagaagtaatgctcgcggtatttaacaagcatcaggatataaccttccatgatgttcaaggttttttgtgtttccaatatcctggaagtgtagatgatggattcccaaccatcacattccattttgaggatgatcttgcactacatgtgtatccccacgagtactttttcgcaaatgggaatgacgtatactgcgtgggattccaaaatggtgcatcacagtcaaaggatggaaaggacattgtgcttatgggagatcttgtcctttccaacaagttggttatctatgacttggaaaatcgagtcattgggtggactgactacaactgttcgtcgagcattaaaattaaggacgacaaaacgggcgcaacatctactgtcaattcacatgacttgtcctcaggatggaagttccactggcacatgtccccggttctattgttggtaacaacggtgtgcagttatttaatatgc

SEQ ID NO: 8 - DNA, 1560 bp, Zea mays
atgctgggtgtggggatggccgcggcggtgctgctcggggccgtggcgttggtgctcgcggacgccgctgcgaggagagcgcactggtggtacagggaggcggctgaggcggtgctggtcggcgccgtggctttggtggtggtggacgccgcggcgcggagggcgcacgggtggtacagggaagcggcgctgggctcggcgcggcgggcgcggctgccgccgggggagatggggtggcccttggtcggcggcatgtgggccttcctccgcgccttcaagtccggcaagcccgacgccttcatcgcctccttcgtccggcggttcggtcgcacgggcgtgtaccgaagcttcatgttcagcagcccgactgtgctggtgacgacggcggaggggtgcaagcaggtgctgatggacgacgacgcgttcgtgacggggtggcccaaggcgacggttgcgctggtcgggccgcggtcgttcgtggcgatgccgtacgacgagcaccggcgcatccgcaagctgactgccgcgcccatcaacggcttcgacgcgctgacggggtacctgcccttcatcgaccgcaccgtcacgtcgtcgctgcgcgcgtgggcggaccacggcggcagcgtcgagttcctgacggagctgcggcgcatgacgttcaagatcatcgtgcagatcttcctgggcggcgcggaccaggccaccacgcgcgcgctggagcgcagctacacggagctcaactacggcatgcgcgccatggccatcaacctgcccggcttcgcgtaccgcggggcgctgcgcgcgcgccgccgcctcgtggccgtgctgcagggcgtgctggacgagcgccgcgccgccagggccaagggggtctcgggcggaggagtggacatgatggaccggctgatcgaggcccaggacgagcgcgggcggcgcctggacgacgacgagatcatcgacgtgctcgtcatgtacctcaacgccggccacgagtcgtccggccacatcaccatgtgggccaccgtgttcctgcaggagaatccggacatgttcgcgagagcaaaggcggagcaggaggcgatcatgaggagtatcccgtcgtcgcagcaggggctgacgctcagggacttcaggaagatggagtacctgtcgcaggtgatcgacgagacgctgcggctcgtcaacatctccttcgtctccttccgtcaggccaccagagacgtcttcgtgaacggatacctcatccccaagggctggaaggttcagctctggtaccggagcgtgcacatggacccacaggtgtaccctgaccccaccaagttcgacccgtcgaggtgggagggccactcgccgagagccggcacgttcctggcgttcgggctgggcgccaggctctgccccggcaacgacctcgccaagctggagatttccgtcttcctccaccacttcctcctcggctacaagctggcgaggacgaaccctaggtgccgggtgaggtacctgccgcacccgaggccggtggacaactgcttggccaagatcaccagagtcggtagctag

SEQ ID NO: 9 - DNA, 1620 bp, Zea mays
atgctgggtgtggggatggccgcggcggtgctgctcggggccgtggcgttggtgctcgcggacgccgctgcgaggagagcgcactggtggtacagggaggcggctgaggcggtgctggtcggcgccgtggctttggtggtggtggacgccgcggcgcggagggcgcacgggtggtacagggaagcggcgctgggctcggcgcggcgggcgcggctgccgccgggggagatggggtggcccttggtcggcggcatgtgggccttcctccgcgccttcaagtccggcaagcccgacgccttcatcgcctccttcgtccggcggttcggtcgcacgggcgtgtaccgaagcttcatgttcagcagcccgactgtgctggtgacgacggcggaggggtgcaagcaggtgctgatggacgacgacgcgttcgtgacggggtggcccaaggcgacggttgcgctggtcgggccgcggtcgttcgtggcgatgccgtacgacgagcaccggcgcatccgcaagctgactgccgcgcccatcaacggcttcgacgcgctgacggggtacctgcccttcatcgaccgcaccgtcacgtcgtcgctgcgcgcgtgggcggaccacggcggcagcgtcgagttcctgacggagctgcggcgcatgacgttcaagatcatcgtgcagatcttcctgggcggcgcggaccaggccaccacgcgcgcgctggagcgcagctacacggagctcaactacggcatgcgcgccatggccatcaacctgcccggcttcgcgtaccgcggggcgctgcgcgcgcgccgccgcctcgtggccgtgctgcagggcgtgctggacgagcgccgcgccgccagggccaagggggtctcgggcggaggagtggacatgatggaccggctgatcgaggcccaggacgagcgcgggcggcgcctggacgacgacgagatcatcgacgtgctcgtcatgtacctcaacgccggccacgagtcgtccggccacatcaccatgtgggccatggcggccaccgcgatggaccatgacggcggcgacgacgtggtcacaccaggggagcttctcggaaactcattaaccctcgcggccggacgtggcgcctacgccgacggtcgctctgtgcgcgcgtcggtcaccgggcgccgccgcatcgtgccgcccgcacctgcctccgacgaccagagatccacggtggaggtggtggggcacaaggcccatggagctgtgccgcagccgggaagcgtcgtcatcgcccgagtaacaaaggttatggctagaatggcctctgcagatatcatgtgtgttgactcaaaggctatcagagaaaagttcactggcatgataaggcagcaagatgttcgtgcaactgaaattgacaaggtggatatgtaccaatcatatcgtcctggggacattgttaaagctatggttctttctcttggtgatgcaagggcatattacctttccactgcgaaaaatgaacttggagtcgtttcagcacaaagcatagctggtggtacattggttccaatcagttggactgagatgcaatgtgatttgaccggtcaaattgagcaaaggaaagttgcaaaggtggaatag

SEQ ID NO: 10 - DNA, 1482 bp, Zea mays
atggatcctaccaagttccgtccgtccagtagccacgacacgacggtgacgacgacgaacgctggcgctcctgtgtggaacgacaacgaggcgctgactgtggggcctcgcggtcccatcctgctggaggactaccacctgatcgagaaggtggcgcacttcgcgcgcgagcgcatcccggagcgcgtggtgcacgcgcgcggcgcctcggccaagggcttcttcgagtgcacccacgacgtgacgtcgctgacgtgcgccgacttcctgcgcgcgcccggcgtgcggacgcccgtgatcgtgcgcttctcgacggtgatccacgagcgggggtccccggagacgatccgcgacccgcgcgggttcgccgtgaagttctacacccgcgagggcaactgggacctgctgggcaacaacttccccgtcttcttcatccgcgacggcatcaagttccccgacgtgatccacgcgttcaagcccaacccgcggtcgcacgtgcaggagtactggcgggtgttcgacttcctgtcgcacctccccgagagcctgcacaccttcttcttcctcttcgacgacgtgggcgtgccgtccgactaccgccacatggaagggttcggcgtgaacacgtacacgttcgtgagcgcggcggggaaggcgcagtacgtgaagttccactggaagccgacgtgcggcgtgcggtgcatcctgacggacgaggaggcggcgctggtcgggggacggaaccacagccacgcgacgcaggacctgtacgactccatcgcggcggggagcttcccggagtggacgctgtacgtgcaggtgatggacccggacacggaggagcagtacgacttcgacccgctggacgacaccaagacgtggccggaggacctgctgccgctcaggcccgtggggaggctggtgctggacaggaacgtggacaacttcttcaacgagaacgagcagctggcgttcgggccggggctggtggtgccagggatctactactcggacgacaagatgctgcagtgccgggtgttcgcctacgccgacacgcagcgctacaggctgggaccaaactacctgatgctgcccgtcaacgcgccgcgctgcgcgcaccacaacaaccactacgacggcgccatgaacttcatgcaccgcgacgaggaggtggactactacccgtccaggcacgcgccgctgcggcaggcggcgccgcccacgccactgccgcccaggccagtcgccgggaggagggagaaggccaccatacgcaagcccaacgacttcaagcagccaggggagaggtaccgctcctgggacgccgaccgacaggaccgattcgtgaggcgattcgccgactcgctcggacacccaaaggtcagccaggagctcaggtccatctggatagacctcctcgccaagtgcgacgcgtcgctggggatgaagattgccacccggctcaacatgaagccaaacatg

SEQ ID NO: 11 - DNA, 780 bp, Glycine max
atggcatgctcagccacctctgcttccctcttctccgcaaacccaacacccctcttctctcccaaaccctctctctccctccaccttaaccctctccccacgcgcccttctccctccctcacgcgcccttctctctccctcacgcgcccttctcactctcgccgctccttcgttgtcaaagcctcctccagcgagcttccgctggttggtaacaccgcaccggatttcgaagcagaggctgtgtttgaccaggagtttatcaacgtgaaactatctgattacattgggaagaaatatgttgtgctcttcttctacccgttggacttcacctttgtctgccccacagaaatcactgctttcagtgaccgacatgcagagttcgaggcactaaatactgagatattgggtgtttcagttgacagtgtgttctcgcaccttgcatggatccaaacagatagaaagtcaggtggccttggtgacttgaactatcctttgatttctgatgtcaccaaatccatatcgaaatcttatggtgttctcattcctgatcagggaattgcattgagaggattgttcattattgacaaggaaggggttattcagcattctaccattaacaacttggcaattggtagaagtgttgacgagacaaagagaactctccaggccttacagtatgtgcaggagaacccagatgaagtttgccctgctgggtggaagcctggggagaagtccatgaaaccagaccctaaacttagcaaagactactttgcagcggtg

SEQ ID NO: 12 - DNA, 624 bp, Zea mays
atgtcgacggtcacttcttgcacctttctgagcctcgggtccccagtgcggccggcagtctcctccgcgagaccggcggcggcggcggtggcttcccgacggaggccccggtccctttctgtgtgctgcgaggccggccggaaaggggatcacaacccaaagacggatctgcatccatttaacatccctgccttcgttctggtgcacccggtggcaccacgtgaggaacggtggcagctggaggaggacgccgacaaggtgaacctgtggttcgaggtgcctgggcagtccaaggatgacctcaccgtggagatcgacgaagacgtgctcgtcatcaagaagaggaaggagctggtgggccagcggagccctgctggcggtggcgtcgcagagtacggctcgcagtcccagcagcagagcaggaggggcaccgcagatggcaaggagctgccggcggcacagcagggccaggccggcgaggtggtctatgcccggatgctcctcccggcggggtacagcagggatggcgtgcaggccgagctcaagtccggcgtgctcagggtcactgtcgtcaaggtcacggagcgagcgcgcaggaagatcgacgtccccattcaagtcaag

SEQ ID NO: 13 - DNA, 1362 bp, Glycine max
atggcctcttcacgccctgccaatgtgggaatccttgccatggacatctacttccctcccacctgcgtcacccaggatgctttggagggtcatgatggggtgagcaaagggaaatatactattgggcttggacaggattgcatggccttctgctctgaggttgaagatgttatctcaatgagcttgacggtagttacttcacttcttgaaaaatttaatgttgatccaaagcaaattggacatttggcggttgggagtgaaactgttattgacaaaagcaaatcaattaagaccttcctgatgcaagtttttgaggcaagtggtaatactgacattgaaggtgttgattcaactaatgcatgctatggaggaacggctgctttgttcaactgtgtgaattgggtggagagtagctcatgggatggacgttatggacttgttgtttgtacagacactgcggtatatgccgaaggacctgctcgtcccactggaggagctgctgcaattgccatgcttgtagggccagatgctcctattgcttttgaaagcaaactcagaggcagtcacatgtctcatgcatatgatttttacaagccaaaccttgctagcgaatatccaattgttgatggaaaactctcacagacctgttatctcatggcacttgattcctgttaccggctttactgtgagaaatttgaaaaattggaggggaggcctttttcaatgtcagattctgattattttgtgtttcattctccatataacaagcttgtgcagaaaagttttggccgactatacttcaatgacttcttgagaaatgccagttttgttgatgaagttgccagggaaacccttgcaccatatgcatccttatctggtgatgagagttatcaaagtcgtgatcttgaaaaggcaaaccagcaagctgcaaaacatctatatgatgcaaaggtgcagcccagcacactaatcccaaagcaagttggtaacatgtacactgcatctctttatgcagcatttgcatctcttcttcacaataagaacagttcgttggtaggtaaacgggtagttatgttttcatatggaagtggtttaacagctacaatgttctccttccagcttcaagagggtcaacatccgtttaacttgtcaaacattgtaacagtgatgaatgtttcggacaagttgaagcagagagttgagattcctcctgaaaagttcgttgaaacattgaagatcatggaacaccgttatgggggtaaggactttgtgacaagcaaggactgtagctacttaactccaggcaccttctatctcaccaatgttgattccatgtacaggagattttatgccaagaaggac

SEQ ID NO: 14 - DNA, 1008 bp, Glycine max
atggagaattcgaccgaagaatcccatctccgatcggaaaactccgtcacttacgagtccccttaccctatctacggcatgtcattctccccctcccacccccaccgcctcgccctcggcagcttcatcgaagaatacaacaaccgcgtcgacatcctctctttccaccctgacaccctttcggtaactccccacccttctctctccttcgaccacccttaccctcccaccaaactcatgttccacccccgcaaaccctccccttcctcttcctccgacctcctcgccacctccggcgactacctccgcctctgggagatccgtgataactccgtggatgccgtctccctcttcaacaacagcaagaccagcgagttctgcgcccccttaacctctttcgactggaacgacatcgaccccaaccgcatcgccacctccagcatcgacaccacctgcaccatctgggacatcgaacgcaccctcgtcgaaacccaactcatcgctcacgacaaggaggtttacgacatcgcctggggagaggccagagtcttcgcctccgtctccgccgacggctccgttagaatcttcgaccttcgcgacaaggagcactccaccatcatctacgagagcccccaccctgacacccctttgctccgcttggcttggaacaaacaggacctgaggtacatggccaccattttaatggacagtaataaagttgtgattttggatattaggtctcccactacccctgttgcggagttagagaggcaccgtgggagtgtgaacgccattgcttgggctcctcatagctccacgcatatttgttctgctggtgatgatactcaggctcttatttgggaattgcccacgcttgcttctcccactgggattgatcccgtctgcatgtactctgctggctgtgaaattaaccagctgcagtggtccgccgcccagcccgattggattgccattgcttttgccaacaagatgcagcttttgaaggtt

SEQ ID NO: 15 - DNA, 999 bp, Synechocystis PCC6803
atgggtcacaaatacgatgtctatggcatgggaaatgccctggtggatatggaatttgaggttaccccagagcagttggccagcctaggcattgataaaggggtgatgaccttggtggaagaggccagggaaaatgaattaattgcccaattggcccaacagcgtggcaagcaaagcagtggcggatcagcggccaatactctggtgtccctggcccaactggggggtacgggtttttatgcctgcaaagtgggtaaggatgaagcgggagccttttacctccaggatttgaacgattgcggcctcgataccaatccccaccatgaaaccgctggcgaaggcattacgggcaaatgcttagtgtttgtcacccccgatgctgaccgcaccatgaatgcttttttgggcattagtggcagtttgtccgtaaccgaaatggattggtctgctttaaaacagtcccaatacctttacctggaaggctatctggtcacttcccccagtgccaaagcggcctgcattgaagccaaggcgatcgccgaacaatccggggtgaaaacctgtctatccctatccgatcccaacatggccaaattttttcaagacggtttaaaggaaatgctggggtcaggggtagatctactattcgccaatgaagcggaagccctggaaatggccggtacttcagacctcaaccaggcgatcgcctactgtaaaagtattgccaaaaactttgccctgaccaggggcggagcaggctcactaatttttgatggtgagaatttattgaccattggcacccccaaagtccaacccatcgatacggtgggagcgggggatatgtacgctgggggatttttatatggcctaacccatggcatggactatgaaaaagctgggcaattggcttcggaaacagccgccaaagtagttacctgttatggtccccgcctagacacagaaattctgcaagagattttacagtctgttcaagcagtt

SEQ ID NO: 16 - DNA, 1146 bp, Zea mays
atgaaggtagagcgcgatctccacatgagccggggcgacggcgaggacagctacgcctccaactcaaggcttcaggagaagtccatactgaagacgaggccggtgctgcacaaggccgtggcggcggcgcacgccttgtcgctctcctcaggaggacccggcggcggcgccatggtcgtcgcggacctcggctgctcgtcggggcccaacacactgctcgtcgtctcggaggtgctagcagcggtggcgatggtggccggcggcagcgcccaaccgcagcacgtgcagttcttcctgaacgacctccccgggaacgactttaacctcgtcttccggtcgctggacctgttgaagaacaagaagctggccgccaaggacaggcgggaggagtcgctgctaccgccgtactacgtggccgggctgccgggctccttctacacccggctgttcccggaccactgcgtccacctcttccactcctcctactgcctcatgtggcgctccaaggtccccgacgagctcgccggtggcgcggtcctcaacgagggccacatgtacatctgggaaaccacgccgcaggcggtggtggccctgtacaggaggcagttccaggaggacatgtcgctgttcctcaggcttcgccacagggagctcgtgcccggtggccacatggtgctggcgttcctgggcaggaagaagagcaaggacgtgcttcgtggcgaggtcagctacacgtgggggctcctcgcgcaggctctccagtccctcgtcaaacagggccgtgtgaagaaggataagctggactccttcaacctgccgttctacgcaccttcgatggacgaggtgagggacgtgatcacgcggagccaggcgttcgacatcacccacatccagctcttcgagtccaactgggatccacacgacgacgacgacgtggagatgaagatggaagaagacgtcgccgccgtccagagcggcgtcaacgtcgccaggtccatcagggccgtgatcgggcccctcatcgcccgccactttggcgagcacatactcgacgatctcttcgagctgcatgccaaaaatgtcgcagtgcacctgcagaaagtgaagaccaagtaccctgtcatcgtcgtttccctccaggcgaaa

SEQ ID NO: 17 - DNA, 687 bp, Saccharomyces cerevisiae
atgggaggtatacttaaaaacccgctagccttatcgccggagcagctggcgcaacaggatccagaaacgcttgaagagtttagaagacaagtttacgaaaatacgcagaaaaatgccaaattgacatcacataaaaggaatataccgggactagataatacaaaagaggagggggagataattggaacatcgagtaccttccttccaaaggacacactgtcactgaaacatgagcaagacatgttggccaagatgacaccagaagagcgagttcaatggaaccaaagaaatttagcagaaaatgaaataacaaaaaagcagttccaagatattcacatagacgagcccaagaccccctaccaaggtgctgtggaccctcacggggaatactacagagtagatgatgatgaagatgaggataatagtgataaaaaaccatgccaggtggcaaacgatgacatcgacgatttgtccctgggcgaaccggaatttgaaatcaaagagaacaaacaaccggattttgagaccaacgacgagaatgatgaagactcaccggaggctagacataagaaatttgaagaaatgagaaagaagcattacgacgtaagggcaattttcaacaaaaagtcccgtgaagcgctaaaggacgaagacgaagacgaagatgatagtacgacaaaagaacca

SEQ ID NO: 18 - DNA, 213 bp, Synechocystis PCC6803
atgtttaaacccagcttacttttgtctttgcttgttgtgggctgtttgctaacggttaccggttgcggtggtggcaatcagagggaggaaggtcaacccactcaacagaacatcgaacagcaagaaaataacaacgggaatgccaataccaatgatgatgataatgacaataacgacgacgatgaggaagatgacaacaaagatggagatgat

SEQ ID NO: 19 - DNA, 1119 bp, Glycine max
atgtctgctgttgagtcagctgaacacaacaacatcagaggagtaccaactcatggtggacgctatgttcagtacaatatctatggcaatctcttcgaagtttccagaaagtatgtccctcctattcgccctgtcggtagaggtgcttatggtattgtttgcgctgctgtaaatgcagagacaggcgaggaagttgccattaagaagattggcaatgcattcgacaacagaatagatgccaaaaggaccttacgggaaattaaacttcttcggcacatggatcatgcaaatattatgtccattaaagatattatacgtcctccacagaaggaaaacttcaatgatgtgtaccttgtttctgagttaatggacacagatctgcatcaaataattcgttccaatcagcaattgactgatgatcattgtcggtattttctgtatcaattgttacgagggctcaaatatgtacattcagcaaatgttttgcaccgagatctaaagcctagtaatttgctattgaatgccaattgtgaccttaagattgcggactttggtcttgctagaaccacatctgaaactgactttatgactgagtatgtggtcactagatggtaccgagctcccgaattgcttcttaattgttcagaatatacagcagccattgatatatggtctgttggttgcatacttggtgaaatcataacaagacaaccgctctttcctggcaaagattatgttcatcagctgagacttatcacagagctgataggttcgccggatgatgccagccttggatttctacgaagtgataatgctcgtagatacgtaaaacagcttccgcagtatccaaaacaaaacttttctgctagatttcccaccatgtctcctggtgcggttgacttgctagagaagatgctcatctttgatccaaacaggcgtattacagttgacgaggcgttgagccacccatacatgtcacctctccatgacatcaatgaggaacctgtttgcaccagacctttcagttttgactttgagcaaccatcattcactgaagaagacatcaaggaactcatctggagagaatctgtgaagttcaatcctgcaaccatatatgtt

SEQ ID NO: 20 - DNA, 1617 bp, Saccharomyces cerevisiae
atgccattgaatgaaaaatacgagaggcctccgcaacctcctccagcatatgatcccaaccataggccgcccagttcttccgagaactctgccgcggcgaatgtcaacgatggccagacgccgtaccattttagacaggatcaatattacaatctaaattccaaaacgagtggggctcctatagggagcttcgacgaggcctttcctactgaaaatgacaacaagcccaggtggaacgattggccatttaccatttttttcctatgcacggtaggcggattcatcgccattgctgccataaccttaagagcgtggtcccaaacctatagcagtacgggttccggaatatacgatggtgtgaatactggtacgctgaacaccaatgctgccatcctgctagttttcgtgtgcattattgccctagtattctccgttttgggcctcacactttgtcgaatttttccgaagcagtttatatattgcggtatggtcatcaacctagtggcttctcttgggactgccatcatgtacatgtctttgaggtactggtctgcaggtattgtgtttttagtcttcacgttcatgaccgcgtggtgctactggggtatgaggtctagaattcccctttctgtggcggtattgaaagtcgtcgtcgatgccatgaagaaatgcccacagatcttctttgtttcatttgttggagcgctagttgctagtgcgtttgggtttctcttctcagcagtcattgtggctacgtacataaagtacgatcctaatagctcgaatggcggctgtgacgtttctggtggcagctgttcgcattccaaattgattggtgttttggttgtggtttttttctgcggctattatatctctgaagtaataagaaacgtcatacattgtgttatttctggcgtctttggcagctggtattatatgtcaaagtctgaccaaggaatgccaaggtggcccgcatttggagcattgaagagagccatgacttactcgttcggttccatttgctttgggtccttgctagtcgctttgatcgacctgttgagacaaatattgcaaatgatcagacacgatgttacctccagcggtggcggtcaaattgccatccagattttgtttatggtgtttgactggattatcggctttctgaagtggctcgccgagtatttcaatcattatgcatactcgttcatcgctctttatggtaaaccgtatttaagagcggccaaagaaacctggtatatgttaagagaaaagggaatggacgccctaatcaatgataatttaattaacattgcactgggtctattttcaatgtttgctagttatatgaccgctttgttcacgttcttgtatctaaggtttacctcgcctcaatacaattcgaacggcgcttacaacggcgccctaatggcattctcatttgttattgctttacagatttgcaacattgcaacagaagccattagatccggtactgcaactttctttgttgctcttggtaatgacccggaggtcttccatcattcttaccctcataggtttgatgagatttttagggcatatcctgatgtactcagaaaattgagtcaccagaatgtg

SEQ ID NO: 21 - DNA, 999 bp, Triticum sp.
atggctgcccagaacaacaaggaggtggatgccctggtggagaagatcaccggcctccacgccgccatcgccaagctgccgtcgctcagcccatccccagccgtcgacgcgctcttcaccgagctggtcacggcctgcgttcccccgagcccagtggacgtgaccaagctcggcccggaggcgcaggagatgcgggagggcctcatccgcctctgctccgaggccgaggggaagctggaggcgcactactccgacatgctcgccgccttcgacaaccctctggatcacctcggcatgttcccctactacagcaactacatcaacctcagcaaactggagtatgagctcctggcgcgctacgtgcccggaggcattgccccggcccgtgtcgcgttcattggctccggcccgcttcccttcagctcctttgtcctcgccgcgcgccatctgcccgacaccatgtttgacaactacgacctgtgtggcacagccaacgaccgtgcgagcaagctgttccgcgcggacacggatgtgggcgcgcgcatgtcgttccacacggccgacgtagcggacctcgccggcgagctcgccaagtacgacgtggtcttcctggccgcacttgttggcatggccgccgaggacaaggcgaaggtgatcgcacacctcggcgcacatatggcggacggtgcggctctcgtcgtgcgcagcgctcacggggcacgcgggttcctgtacccgatcgtcgacccccaggacatcaccctaggcgggttcgaggtgctggccgtgtgccacccagacgacgacgtggtgaactccgtcatcatcgcacagaagtccaaggacgtgcatgtgagtggacttcacagtgggcgtgctgtgggtggacagtctgctcgcggcacggtgccggtggtcagcccgccgtgcaggttcggtgagatggtggcggaggtgacgcagaagagagaggagttcgccaacgccgaagtggccttc

SEQ ID NO: 22 - DNA, 1002 bp, Zea mays
atggctggcgcgcaggagtccctctctctggtgggcacgatgcgcggccacaacggcgaggtgacggcgatcgcgaccccgatcgacaactcgccgttcatcgtctcctcctcccgcgacaagtccctgctggtgtgggacctgaccaacccggtccactccaccccggaatccggcgccaccgccgactacggcgtccccttccgccgcctcaccggccactcccacttcgtccaggacgtcgtcctcagttccgacggccagttcgccctttccggctcctgggatggagagctccgcctctgggacctctccaccggcctcaccacccgccgcttcgtcggccacgagaaggatgtcctctccgttgccttctccgttgacaaccgccagatcgtctccgcgtcccgcgacaagaccattaagctctggaacaccctcggtgagtgcaagtacaccatcggaggtgacctcggtggcggcgagggccacaacgggtgggtctcctgcgtcaggttctccccaaacacatttcagcccaccattgtctccggttcctgggaccgcaccgtcaaggtctggaaccttacgaactgcaagctgcgctgcactctcgatggtcatggcggatatgttaacgccgtcgccgtgagccccgatggatcgctctgcgcctccggcgggaaggacggtgttactctgctgtgggatttggctgaggggaagaggctgtactcgctggatgcgggctccatcatccactccctctgcttctcgcccaaccgctactggctctgcgctgcgacccaggactctgttaagatctgggaccttgagtcgaagcacgtcgtgcaggacctcaagcctgacatccagatctccaagaaccagatcctgtactgcacaagcttgagctggagcgccgatggaagcaccctctacactggctacaccgatgggtctatcagggtctggaagatctctggatatggctacgcaggc

SEQ ID NO: 23 - DNA, 603 bp, Saccharomyces cerevisiae
atgtctagtgccatcgttgctaaactgaacaaagaggacatcattaaggacaccgtcaaggacttggcgttcgagattcttggtgagttgtcggtttcgtacgttgattctgacgacatcaaattgggcaatcccatgccaatggaagctacgcaagccgctcctaccatcaagttcacgcccttcgataaaagccaattgtcagccgaggataaactcgctttgttgatgactgacccggacgcgccctctagaacagaacataagtggtcggaggtttgtcactacatcatcactgatattcctgtcgagtacggacctgggggagatattgccatttcggggaagggtgtcgtgagaaataactatattgggcctggtccaccaaagaactccggttatcacagatacgtgttcttcctgtgcaagcaacccaagggagcggattcgtccacttttaccaaggttgagaacattatctcctgggggtacggtactcctggtgcgggtgcctatgactatatcaaggagaacaatctgcaactggtcggtgccaattattacatggtcgaaaacacgaccgttgatttcaaccatgacatg

SEQ ID NO: 24 - DNA, 366 bp, Oryza sativa
atggcgggcgtgtgggtgttcaaggacggcatcgtgcggcgcgtggagaaccccggcagcgaggaatcgtcgtcggcgggggacggcggcggaggcgggcggcggaaggtgctggtgcacgtgccgagcggggaggtggtggcgtcatacgaggtgctggagcggcggctgcgggagctcgggtgggagaggtacctcaccgacccgtgcctcctgcagttccaccagcgctccaccgtgcacctcatctctgtcccccgcgacttctccaagttcaagctcgtccacatgtacgacatcgtcgtcaagacccgcaacgtcttcgaggtccgcgacgccgctgcccccgccgtctcaccggcgacc

SEQ ID NO: 25 - DNA, 354 bp, Oryza sativa
atggcaggggtgtgggtgtttgaggatgggatggtgaggagggcagatagcgaggcgccgtcgagagggcgcggtgtcggtggtggaggtgggggagggaaggtgcttgtgcacgtgccgagcagcgaggtggtgacgagctacgaggttctggagaggcggctgcgggagctcgggtgggagaggtacctcaacgacccgtgcctcctccagttccaccagcgctccaccgtccacctcatctccgtgccccgcgacttctcccgcctcaagctcgtccacatgtacgacgtcgtcgtcaagacccgcaacgtcttcgaggtccgtgacgccgccaccactgcttccccgcca

SEQ ID NO: 26 - DNA, 957 bp, Zea mays
atggcgatcaagcggaccaaggcggagaagaagatcgcgtacgacaagaagctgtgcagcctgctcgatgagtacaccaaggtgctcatcgccctcgccgacaacgtcggttccaagcagctccaggacatccgccggggcctcaggggcgactcggtggtgctcatggggaagaacacgctcatcaggcgctgcatcaaggtgtacgccgagaagaccggtaaccacacctttgacccgctcatggacctcctggtcggcaacgtaggcctcatcttcaccaagggagacctcaaggaggtgcgcgaggaggtcgccaagtacaaggtgggggctcctgctcgtgttggtctggttgctccagttgatgttgttgtgccccctggcaacactgggctggatccgtcccagacctctttcttccaggtgcttaacatccccaccaagattaacaagggtaccgtggaaattatcacccctgtggagctgatcaagaagggtgaaaaggtgggctcgtccgagtctgccctgctcgccaagctgggtatccgccccttctcgtacggcctccaggtcaccagtgtctacgaggatgggtccgtgttcagccctgaggtgctcgacctttctgaggaggacctgattgagaagtttgccactggtgtctccatggttgcctccctgtcactggcgatctcgtacccgacccttgctgctgtgccccacatgttcatcaatgggtacaagaatgtgcttgctgttgctgtcgagacagactactcatacccacatgctgacaagatcaaggagtacctcaaggacccaagcaagtttgctgttgccgccccggttgctgctggggactctggcgctgcagctgctcccaaggaagaagagaaggctgcggagcctgaagaggagtcggacgaggaaatgggcttcagcctcttcgacgac

SEQ ID NO: 27 - DNA, 1044 bp, Zea mays
atggagaacggcggcgctaaaggcgacgtccccgacgatgcgaacgagcattgccctggaacacagtctgaggaggcaggcaaggcagatgcctgcgctgggtgccccaaccagcagatttgcgccaccgcccccaaggggcctgatcccgatgtggttgctattgttgagaggttggctactgtgaaacacaaattgttagttttgtctggaaagggaggtgttggaaaaagcacattctcagcccaactctcgtttgccctagctgaaatggaccatcaggttggccttcttgacatagatatatgtggccctagcatccctaaaatgctaggccttgaaggacaagatattcatcaaagcaaccttggctggtcacctgtgtatgttgagtccaaccttggtgtgatgtcgattggtttcatgctgcctaatccagacgatgctgtcatatggaggggtccccgcaagaatggactgatcaagcagttcttaaaggatgtcgattggggggagattgactatcttgttgtggacgctcctccaggaacatctgatgagcacatctcgatcgtgcaataccttcaagccacaggaatcgatggcgcgataatcgtcacgactccgcagcaggtctctctgattgatgtgaggaaggagataaacttctgcaagaaggtcggtgttcctgtcctaggtgttgtggagaacatgagtggtctgaggcagccgctttcagacctcagattcatcaagtcagatgaggcgggcaagacagatgcgacagaatgggccctgaactacatcaaggagaaagcccctgagcttctgtcggtcatggcctgcagtgaggtatttgatagcagcaagggaggcgccgagaagatgtgccatgaaatgggggttcctttccttggtaaggtgcccatggatccgcagctgtgcaaggcggctgaggaagggaggtcatgctttgctgatcagaaatgcagtgccagtgcgccggctcttcgaaacatcgtgaagaagctgatcaaggccgag

SEQ ID NO: 28 - DNA, 588 bp, Arabidopsis thaliana
atgggaagaagaaaaatcgagatcaagcgaatcgagaacaaaagcagtcgacaagtcactttctccaaacgacgcaatggtctcatcgacaaagctcgacaactttcgattctctgtgaatcctccgtcgctgttgtcgtcgtatctgcctccggaaaactctatgactcttcctccggtgacgacatttccaagatcattgatcgttatgaaatacaacatgctgatgaacttagagccttagatcttgaagaaaaaattcagaattatcttccacacaaggagttactagaaacagtccaaagcaagcttgaagaaccaaatgtcgataatgtaagtgtagattctctaatttctctggaggaacaacttgagactgctctgtccgtaagtagagctaggaaggcagaactgatgatggagtatatcgagtcccttaaagaaaaggagaaattgctgagagaagagaaccaggttctggctagccagatgggaaagaatacgttgctggcaacagatgatgagagaggaatgtttccgggaagtagctccggcaacaaaataccggagactctcccgctgctcaat

SEQ ID NO: 29 - DNA, 972 bp, Glycine max
atggaacgaagtggcggaatggtaactgggtcgcatgaaaggaacgaacttgttagagttagacacggctctgatagtaggtctaaacccttgaagaatttgaatggtcagagttgtcaaatatgtggtgataccattggattaacggctactggtgatgtctttgtcgcttgtcatgagtgtggcttcccactttgtcattcttgttacgagtatgagctgaaacatatgagccagtcttgtccccagtgcaagactgcattcacaagtcaccaagagggtgctgaagtggagggagatgatgatgatgaagacgatgctgatgatctagataatgagatcaactatggccaaggaaacagttccaaggcggggatgctatgggaagaagatgctgacctctcttcatcttctggacatgattctcaaataccaaacccccatctagcaaacgggcaaccgatgtctggtgagtttccatgtgctacttctgatgctcaatctatgcaaactacatctataggtcaatccgaaaaggttcactcactttcatatgctgatccaaagcaaccaggtcctgagagtgatgaagagataagaagagtgccagagattggaggtgaaagtgccggaacttcggcctctcagccagatgccggttcaaatgctggtacagagcgtgttcaggggacaggggagggtcagaagaagagagggagaagcccagctgataaagaaagtaaacggctaaagaggctactgaggaaccgagtttcagctcagcaagcaagggagaggaagaaggcatacttgattgatttggaaacaagagtcaaagacttagagaagaagaactcagagctcaaagaaagactttccactttgcagaatgagaaccaaatgcttagacaaatattgaagaacacaacagcaagcaggagagggagcaataatggtaccaataatgctgag

SEQ ID NO: 30 - DNA, 330 bp, Zea mays
atggctgctacaaagcttcaggccctttggaaccacccggctggccccaaaaccattcatttctgggcgccaacattcaaatggggcatcagcattgccaacatagccgactttgctaaaccacctgaaaagatatcttaccctcagcaagttgctgttgcatgcactggactcatttggtcaaggtacagcttggttatcacaccgagaaactggaaccttttcagtgttaacgttgcaatggcgggtacaggcctgtatcagctttcacggaagatcaggcaagattacttatccgatgagaaagatgctgcttcacaactggaagca

SEQ ID NO: 31 - DNA, 306 bp, Oryza sativa
atgaggccgactggtcgtcgttcttctccgccggtggctgctgctcttgccctgcttctcctcctcgtcctcttcttcttctcccactgcgcctcagctgctcgcccactgccagcatcagcagcagcagagctagtgcttcaggatggcgccaccggcaatggcgacgaggtttccgagttgatgggagcagctgaggaggaagcagcaggattatgcgaggaggggaacgaggagtgcgtggagaggaggatgcttcgcgacgcccacctcgactacatctacacgcagaagaggaacaggcct

SEQ ID NO: 32 - DNA, 501 bp, Saccharomyces cerevisiae
atgtctactattccctcagaaatcatcaattggaccatcttaaatgaaattatatctatggatgacgatgattccgatttttctaaaggtctaattattcaatttatcgaccaggcacaaacaacttttgctcaaatgcaacgacagctggacggtgaaaaaaatcttaccgaattagacaatctgggccattttttaaagggttcttctgctgcattaggcttacaaagaattgcctgggtttgtgaaagaattcaaaacttgggaagaaaaatggaacatttcttccccaacaagaccgaattggtcaacactctgagcgataaatcgattattaatggaatcaatattgatgaagatgacgaggaaataaagatacaagtggacgataaagacgaaaattccatatatctcatcttgatagcaaaagctttgaaccagtctaggttggagttcaaactggcgagaattgagttatctaaatattacaacacaaaccta

SEQ ID NO: 33 - DNA, 1053 bp, Glycine max
atggggagatgggtgaattgtaggggcaatatattcccattcctggggatggtgatggctatgctagctcaaagtggaagcatggtggtaatcaaagttgccatgactgatggaataaacaaatatgtcatggttgtgtactctttggcactctccaccatcttgcttcttccctttgccttgtttcttcacaggtcagagcgtcctcctcttacattctctgcactttgcagtttcttcttgcttgcattctttgggagttcaggacagataatggcttatgttggcatagatctaagctctcctactcttgcatcagccatgcttaatctcattccagctttcacttttatacttgctctcatttttaggatggaagaagtacattggaaacattccagcagccaagccaagttcttaggaacaatagtatcaattgctggagcatttgttgtgatactgtacaagggcccacccatctttaagacacatttgtcaaactcttcgaacaaatttctattttcgcagcaactaaactggatcttgggagggatgttctgtgctggcgattctatagtttgttccctgtggtacatatatcaggcttcggttgcatacaaataccctgctgtaacaaccatagtattcttccaactcttattttcaaccattcagtgtggagtatttgccttaattgtagttcaagatcaaacggaatgggagctgaagttagatattgggttgattggcattgtatatcaggcaattgctgcaacattgatacgttacatcttatgcacgtggtgcgtccttaaagctggacctctgttctgttctatgttcaagcctgtggcaatcatcttctccgttttcatgggtgcaatttttctcggggatgacttaagccttggaagtttgattggtgcggttataatcgtgataggattttatgccgtactgtggggaaaatccatagaagataacaagattgagaaaggggttgagaacttggagtcatcgtgccataatgttcctttattacaaaataggacg

SEQ ID NO: 34 - DNA, 1047 bp, Saccharomyces cerevisiae
atgagcaagacagccgtgaaagattctgctacagaaaaaaccaagctaagtgaaagcgaacagcactacttcaattcgtacgatcactatggtattcacgaagagatgcttcaagatactgttcgtaccttatcttacagaaacgcaattatccaaaataaggatctttttaaggacaagattgttttagacgtcggttgcggtaccggtattttatccatgtttgccgctaaacacggtgcgaagcatgttatcggtgttgatatgtcaagcattattgagatggcgaaggaattggtagagttgaacggattcagcgacaagatcaccttgctaagaggcaagttggaggacgttcatttaccctttcctaaagttgacatcataatttctgaatggatgggttactttctactatacgagtccatgatggacaccgttctttacgctagagaccactatttggtagaaggcggtctgatctttcccgacaagtgctccattcatttggccggtttggaagactctcagtataaagacgagaagttgaactactggcaagacgtttacgggtttgattattcgccatttgttccgttggtcttacacgagcccatcgtcgacaccgtggaaagaaacaatgtcaacaccacctcagacaaattgatcgaatttgatttaaatacagtaaaaatatcagatctagcgtttaagagtaactttaaattgacggccaagagacaagatatgattaatggtatagtcacctggttcgacattgttttccctgcaccaaagggtaagagacctgttgagttctccactggtcctcatgctccatacactcactggaagcaaacaatattttatttccctgatgatctagatgctgaaactggtgacaccattgaaggtgaattggtttgctctccaaacgagaagaataacagagatctaaatatcaaaatttcttacaagttcgaatcgaatggcatcgacggtaattcaagaagcagaaaaaacgaaggttcttatttaatgcattaa

SEQ ID NO: 35 - DNA, 1722 bp, Zea mays
atggcatactttcctgatgaagtagtagggtatatccttggttatgtaacgtcgcatcaggaccgcaacgcggtgtccttggtctgccgggcatggtacgacattgagcgtcatggccgccattcggtgcttgtaagaaactgttatgcggtgtgcccagagcgtgtacatatgcggtttcccaacatgcgcgcactgagcttgaagggcaaaccgcactttgctgaattcaaccttgtcccggcgggttggggtgccactgcaaatccatgggtggatgcgtgcgcccgtgcatgcccaggtcttgaggagctccggctgaagtttatggttgtgactgatgaatgcctcaagttgctttctctatcctttaccaactttaaatcacttgtccttgtttgttgtgaggggttcagtactaccgggcttgctaatattgccaccaattgcaggtttcttaaggaactagacttacaaaagagttgtgtgaaacatcaagaccatcagtggattaattgttttcccaagtcttcaacatcactagaatgcttgaatttttcctgcttgactggggaggtgaatgctgttgcactggaggaacttgttgcaaggagtccaaatcttaaaagtttaaggctgaatcttgcagttccatttgatgttttgtccagaatcctttctcgcacacctaagctagaggatttaggcacaggatcctttttacaaggcaatgaccctgctgcatatgccagtttatgtagagctcttgagaactgcacttcactgaagagtatatctggtttttgggatgctccaggcttttatgttcaaggaattttgtcaaattgcaagatcagaaatcttacatgcttgaatctcagctatgctacattgattcagagtacccagcttatcggtattattcgtcattgtaagaagctccatgtcttatgggtgttagatcacattggtgatgagggcttgaaggctgtgtccttttcttgtcctgatcttcaggagttgagggtatatccaagtgttgttgcaccaagaggtactgtgacggaggaagggttggtagccctatcttcgtgtcggaagttacagcgtgtgctcttctcttgtgttcgaatgacaaacacggcgctgatgacaatagcaaggtactgcccacggctaacgtccttcagactatgcattcgcaagcccaggtcagcagacgccgtcacaggacagccactggacgaaggctttggggcgatagtgcggtcctgcagaggcctgaggcgtcttgccatgtccggcctcctcacggacagcgtgttcctgtacatcggcatgtacgcggagaagctggagatgctctcggtaacgttcgcgggggacaccgacgacggcatggtctatgtgctcaatggctgcaggaacctcaagaagctggtaatcaaggagagccccttcggtgacgccgctctcctcgcgggcgcgcacaggtacgagtcaatgcgcagtctctggatgtcgtcctgccagatcaccctgggtggctgcaaggcccttgcggccaccatgccaaacatcaatgtcgaggtcatcggtggggcgagctttggtgcgatggatggtggtgtcagcggtgcgaggaaggtggacatgctctacctctaccgtactctggctggacctagatgcgatacgccaggattcgtctcgatattgtag

SEQ ID NO: 36 - DNA, 411 bp, Saccharomyces cerevisiae
atgaacgatggtcaattcttgttccaacgtaacgatcccatcatattatatacatttttgcttaaaagtaattatatagtctttcgcagcatagatgaaaggctatgtgatttcgttttttatattgaccacttcttgaacaaacgaatcagttatcgcataccaattctaataagaaacaacaacacaaacattttaaacaactgtccaagcagctttcctcctttggttgaccttgttggacatagactggttgctgctgaggataaccctgttgcggtggaccttgttgataacaaccttgttgtggtggaccttgttgataataaccttgctgtgggggtccttgttggtagtaaccttgttgttggctcattagttttcgctcttttgacttgtttcgaagacgga

SEQ ID NO: 37 - DNA, 723 bp, Zea mays
atggcatcctgtggcgccggcgacgccgcgtcagcgcagccgagggccagtatctcgcacgtcatcttcgatatggacggccttctcctcgacacggagggcttctacacggaggtgcaggagaagatcctagcgaggtacgacaaggtgttcgactggtcgctcaaggccaagatgatgggcaagaaggcggccgagtctgcccgcatcttcgtcgacgagtgcggcctcaacggcctcctcacccccgagcagttcctcgaggagcgcgagagcatgctccaagcgctcttcccatcctgtaccaagctgccaggagtactacgtttggtgcatcatcttcatgccaacggagtaccaatggctgttgcgacaggatcccataaacgccattttgcactgaagacccagaatcaccaggaaatgttttccctgatgcatcacgttgtcatgggtgatgatccagaggtgaaggctggcaaaccatcccctgatatttttcttgctgctatgaggaggtttgagggcggcgtagaacctagcaagtgcttggtttttgaagacgctccttcgggtgtagctgcagcgaaaaatgccggaatgtctgtagtgatggtcccggatccaaggctggatgtttcgtatcacaaaggagctgaccaagttctcagttcattgctggacttcaaacctgccgaatggggcttgccggcatttaaagag

SEQ ID NO: 38 - DNA, 831 bp, Zea mays
atggaggccgtggcgcgcagcggcgactacctttggcggttcgtggcggagacagagtggtacaacgaggttgtcctcagcgccgtggcgccaggcgactggtggcgcggcctgccgcacccggtgcagtcatggatgcgcaactgcgtcggcggttacctcctctacttcatctctggtttcctctggtgcttcgtcatctactactggaagcgccacgcctacatccccaaagatgccatccccacaaatgaagctatgaagaagcaaatagttgtagcatcgaaggctatgcctttttactgcgctcttccaactttatctgaatatatgattgagagcggatggactcggtgctacttcaatatcagcgaaattggtttttctatgtacctctgttatatggctatgtatctcatctttgtggagtttggaatttactggatgcacagggagttgcatgacataaaaccattatacaaatatctgcatgcaacccaccatatttacaacaaggagaataccttgtctccatttgctggacttgcatttcatccactggatggtattctgcaagcaataccgcatgtgttcgcgctcttcctctttccaacacacttcaggacacatattgcactcttgttcctagaggctgtgtggacaacaaacatccacgactgcattcatggcaagatatggccagtcatgggcgctggatatcacaccattcaccatacgacttaccgccacaactatggccactacaccatctggatggactggatgtttggtacgctcaatgagccagaagatatcctcaagaaggcttag

SEQ ID NO: 39 - protein, 243 aa, Zea mays
MASADLLRKEEEFYSSLFDSAKGDGVKSRSQVIERKIESLEDMATKVSNRRSRRWLNDRLLIELVPRLHVEEIKGLFAPPPWGEELPLSAFCRTSVSDWEAFRSIDMDAEARLMQHMKRSSEKQRTHVDEDELIALNAWRRIGRQTREAIKKKFLPDLLQIYEEQVRAFIEGTGDSDVLVLNVQDPFQRLLLHGVCEFYNVTSMTTSSVRDGKPWKSTTIKKRHVTGLPPRITLVSFLRMRKN

SEQ ID NO: 40 - protein, 328 aa, Synechocystis sp.
MSENFAVATPVRVGIVGTGYAAQRRAEVFRGDRRSQLVSFWGNSEANTAKFADTFGVRPQQSWQALINDPEIDLVLIATINQLHGAIAEAALQAGKHVVLEYPLALTYAMGKKLQQLAREKGKLLHVEHIELLGGVHQAIRQNLGKIGEVFYARYSTIMGQNPAPQRWTYHHQQFGFPLVAALSRISRFTDLFGTVQQVDAQCRFWDQPNPEYFRACLATAYLQFNNGLKAEVIYGKGEVFHQNERIFTLHGDRGTLIFVGETGRLIQGQTETEITVGSRRGLFRQDTEAVLDYLTTGKPLYVDLEASLYALEVADLCAQACGYKVEN

SEQ ID NO: 41 - protein, 355 aa, Zea mays
MATAEVVVANGSGGADTKTAFKEIYSKLKQEMLEDPAFEFTDESLQWIDRMLDYNVLGGKCNRGLSVVDSYKILKGVDVLSKEETFLACTLGWCIEWLQAYFLVLDDIMDNSQTRRGQPCWFRVPQVGLIAVNDGIILRNHISRILQRHFKGKPYYVDIIDLFNEVEFKTASGQMLDLITTHEGEKDLTKYNLTVHRRIVQYKTAYYSFYLPVACALLLAGENLENFVDVKNILVEMGTYFQVQDDYLDCFGDPEFIGKIGTDIEDYKCSWLVVQALERAAENQKSILFENYGKSDPACVAKVKDLYKELKLEEVFHEYERESYNKLIADIEAQPSKAVQTVLKSFLHKIYKRDK

SEQ ID NO: 42 - protein, 631 aa, Zea mays
MGGGADEVVKVVDSEDGEGEEEEAEAAAAEGSSKETPMLPRMPVRVLLAEGDDSTRHVISALLRKCGYRVAAASDGVKAWDILKEKSFNIDLVLTEVELPLMSGFLLLSTIMEHDASKNIPVIMMSSHDSVSMVFKCMLKGAADFLVKPLRKNELRNLWQHVWRKQLANGGPDVQHIQEENLAERMEQKTGVTKADNLNSDGPHKNRECSEQESDAQSSCTRSELEAESKQTNNILEYMQSTERHLFIRSHKDLELNGETKTRTKGNNLIPTREDDLLPKKRTCLNDNDSERTSRDMELVHIMDNQQKHDTQRDVDTMRTTSRGNDEKNSIPAHQLELSLRRTDYGKLENHEKNDRRTLNHSTSSAFYLYNCRTASSLGNAGDGQLCSTSETLVDVENRNGDLAALSEDTTETNRPPIRVVPFPVPVQCFTFDGQPFWNGTPVASPFYPQSAPPIWNSKTPTWQESTPQATSLPQKSQQNEPVEMDAKPVENAEEQFVVGPPSASGKQLHVEIPKDGLRHISPVTGESGISTVLDSTRNTLSSSGCDSTSNRITASTEPSSNVYRGVPETARAEGLRHLSQREAALNKFRLKRKDRCFEKKVRYQSRKLLAEQRPRVKGQFVRQDHSIQGS

SEQ ID NO: 43 - protein, 261 aa, Glycine max
MTASKDRENFVYIAKLAEQAERYEEMVESMKNVANLDVELTVEERNLLSVGYKNVIGARRASWRILSSIEQKEETKGNELNAKRIKEYRQKVELELSNICNDVMRVIDEHLIPSAAAGESTVFYYKMKGDYYRYLAEFKSGNEKKEAADQSMKAYESATAAAEADLPPTHPIRLGLALNFSVFYYEILNSPERACHLAKQAFDEAISELDTLNEESYKDSTLIMQLLRDNLTLWTSDIPEDGEDAQKVNGTAKLGGGEDAE

SEQ ID NO: 44 - protein, 347 aa, Synechocystis sp.
MTVSEIHIPNSLLDRDCTTLSRHVLQQLNSFGADAQDLSAIMNRIALAGKLIARRLSRAGLMADVLGFTGETNVQGESVKKMDVFANDVFISVFKQSGLVCRLASEEMEKPYYIPENCPIGRYTLLYDPIDGSSNVDINLNVGSIFAIRQQEGDDLDGSASDLLANGDKQIAAGYILYGPSTILVYSLGSGVHSFILDPSLGEFILAQENIRIPNHGPIYSTNEGNFWQWDEALRDYTRYVHRHEGYTARYSGALVGDIHRILMQGGVFLYPGTEKNPDGKLRLLYETAPLAFLVEQAGGRASDGQKRLLDLIPSKLHQRTPAIIGSAEDVKLVESFISDHKQRQGN

SEQ ID NO: 45 - protein, 491 aa, Zea mays
MAPSPPRRAALLLTTVLLLAAAGSTPGATAAGIFQVRRKFTAGVGGGAGANISALRAHDGTRHGRLLAAADLPLGGLGLPTDTGLYYTEIKLGTPPKHYYVQVDTGSDILWVNCITCEQCPHKSGLGLDLTLYDPKASSTGSMVMCDQAFCAATFGGKLPKCGANVPCEYSVTYGDGSSTIGSFVTDALQFDQVTRDGQTQPANASVIFGCGAQQGGDLGSSNQALDGILGFGEANTSMLSQLTTAGKVKKIFAHCLDTIKGGGIFSIGDVVQPKVKTTPLVADKPHYNVNLKTIDVGGTTLQLPAHIFEPGEKKGTIIDSGTTLTYLPELVFKEVMLAVFNKHQDITFHDVQGFLCFQYPGSVDDGFPTITFHFEDDLALHVYPHEYFFANGNDVYCVGFQNGASQSKDGKDIVLMGDLVLSNKLVIYDLENRVIGWTDYNCSSSIKIKDDKTGATSTVNSHDLSSGWKFHWHMSPVLLLVTTVCSYLIC

SEQ ID NO: 46 - protein, 519 aa, Zea mays
MLGVGMAAAVLLGAVALVLADAAARRAHWWYREAAEAVLVGAVALVVVDAAARRAHGWYREAALGSARRARLPPGEMGWPLVGGMWAFLRAFKSGKPDAFIASFVRRFGRTGVYRSFMFSSPTVLVTTAEGCKQVLMDDDAFVTGWPKATVALVGPRSFVAMPYDEHRRIRKLTAAPINGFDALTGYLPFIDRTVTSSLRAWADHGGSVEFLTELRRMTFKIIVQIFLGGADQATTRALERSYTELNYGMRAMAINLPGFAYRGALRARRRLVAVLQGVLDERRAARAKGVSGGGVDMMDRLIEAQDERGRRLDDDEIIDVLVMYLNAGHESSGHITMWATVFLQENPDMFARAKAEQEAIMRSIPSSQQGLTLRDFRKMEYLSQVIDETLRLVNISFVSFRQATRDVFVNGYLIPKGWKVQLWYRSVHMDPQVYPDPTKFDPSRWEGHSPRAGTFLAFGLGARLCPGNDLAKLEISVFLHHFLLGYKLARTNPRCRVRYLPHPRPVDNCLAKITRVGS

SEQ ID NO: 47 - protein, 199 aa, Zea mays
MAATAMDHDGGDDVVTPGELLGNSLTLAAGRGAYADGRSVRASVTGRRRIVPPAPASDDQRSTVEVVGHKAHGAVPQPGSVVIARVTKVMARMASADIMCVDSKAIREKFTGMIRQQDVRATEIDKVDMYQSYRPGDIVKAMVLSLGDARAYYLSTAKNELGVVSAQSIAGGTLVPISWTEMQCDLTGQIEQRKVAKVE

SEQ ID NO: 48 - protein, 494 aa, Zea mays
MDPTKFRPSSSHDTTVTTTNAGAPVWNDNEALTVGPRGPILLEDYHLIEKVAHFARERIPERVVHARGASAKGFFECTHDVTSLTCADFLRAPGVRTPVIVRFSTVIHERGSPETIRDPRGFAVKFYTREGNWDLLGNNFPVFFIRDGIKFPDVIHAFKPNPRSHVQEYWRVFDFLSHLPESLHTFFFLFDDVGVPSDYRHMEGFGVNTYTFVSAAGKAQYVKFHWKPTCGVRCILTDEEAALVGGRNHSHATQDLYDSIAAGSFPEWTLYVQVMDPDTEEQYDFDPLDDTKTWPEDLLPLRPVGRLVLDRNVDNFFNENEQLAFGPGLVVPGIYYSDDKMLQCRVFAYADTQRYRLGPNYLMLPVNAPRCAHHNNHYDGAMNFMHRDEEVDYYPSRHAPLRQAAPPTPLPPRPVAGRREKATIRKPNDFKQPGERYRSWDADRQDRFVRRFADSLGHPKVSQELRSIWIDLLAKCDASLGMKIATRLNMKPNM

SEQ ID NO: 49 - protein, 260 aa, Glycine max
MACSATSASLFSANPTPLFSPKPSLSLHLNPLPTRPSPSLTRPSLSLTRPSHSRRSFVVKASSSELPLVGNTAPDFEAEAVFDQEFINVKLSDYIGKKYVVLFFYPLDFTFVCPTEITAFSDRHAEFEALNTEILGVSVDSVFSHLAWIQTDRKSGGLGDLNYPLISDVTKSISKSYGVLIPDQGIALRGLFIIDKEGVIQHSTINNLAIGRSVDETKRTLQALQYVQENPDEVCPAGWKPGEKSMKPDPKLSKDYFAAV

SEQ ID NO: 50 - protein, 208 aa, Zea mays
MSTVTSCTFLSLGSPVRPAVSSARPAAAAVASRRRPRSLSVCCEAGRKGDHNPKTDLHPFNIPAFVLVHPVAPREERWQLEEDADKVNLWFEVPGQSKDDLTVEIDEDVLVIKKRKELVGQRSPAGGGVAEYGSQSQQQSRRGTADGKELPAAQQGQAGEVVYARMLLPAGYSRDGVQAELKSGVLRVTVVKVTERARRKIDVPIQVK

SEQ ID NO: 51 - protein, 454 aa, Glycine max
MASSRPANVGILAMDIYFPPTCVTQDALEGHDGVSKGKYTIGLGQDCMAFCSEVEDVISMSLTVVTSLLEKFNVDPKQIGHLAVGSETVIDKSKSIKTFLMQVFEASGNTDIEGVDSTNACYGGTAALFNCVNWVESSSWDGRYGLVVCTDTAVYAEGPARPTGGAAAIAMLVGPDAPIAFESKLRGSHMSHAYDFYKPNLASEYPIVDGKLSQTCYLMALDSCYRLYCEKFEKLEGRPFSMSDSDYFVFHSPYNKLVQKSFGRLYFNDFLRNASFVDEVARETLAPYASLSGDESYQSRDLEKANQQAAKHLYDAKVQPSTLIPKQVGNMYTASLYAAFASLLHNKNSSLVGKRVVMFSYGSGLTATMFSFQLQEGQHPFNLSNIVTVMNVSDKLKQRVEIPPEKFVETLKIMEHRYGGKDFVTSKDCSYLTPGTFYLTNVDSMYRRFYAKKD

SEQ ID NO: 52 - protein, 336 aa, Glycine max
MENSTEESHLRSENSVTYESPYPIYGMSFSPSHPHRLALGSFIEEYNNRVDILSFHPDTLSVTPHPSLSFDHPYPPTKLMFHPRKPSPSSSSDLLATSGDYLRLWEIRDNSVDAVSLFNNSKTSEFCAPLTSFDWNDIDPNRIATSSIDTTCTIWDIERTLVETQLIAHDKEVYDIAWGEARVFASVSADGSVRIFDLRDKEHSTIIYESPHPDTPLLRLAWNKQDLRYMATILMDSNKVVILDIRSPTTPVAELERHRGSVNAIAWAPHSSTHICSAGDDTQALIWELPTLASPTGIDPVCMYSAGCEINQLQWSAAQPDWIAIAFANKMQLLKV

SEQ ID NO: 53 - protein, 333 aa, Synechocystis PCC6803
MGHKYDVYGMGNALVDMEFEVTPEQLASLGIDKGVMTLVEEARENELIAQLAQQRGKQSSGGSAANTLVSLAQLGGTGFYACKVGKDEAGAFYLQDLNDCGLDTNPHHETAGEGITGKCLVFVTPDADRTMNAFLGISGSLSVTEMDWSALKQSQYLYLEGYLVTSPSAKAACIEAKAIAEQSGVKTCLSLSDPNMAKFFQDGLKEMLGSGVDLLFANEAEALEMAGTSDLNQAIAYCKSIAKNFALTRGGAGSLIFDGENLLTIGTPKVQPIDTVGAGDMYAGGFLYGLTHGMDYEKAGQLASETAAKVVTCYGPRLDTEILQEILQSVQAV

SEQ ID NO: 54 - protein, 382 aa, Zea mays
MKVERDLHMSRGDGEDSYASNSRLQEKSILKTRPVLHKAVAAAHALSLSSGGPGGGAMVVADLGCSSGPNTLLVVSEVLAAVAMVAGGSAQPQHVQFFLNDLPGNDFNLVFRSLDLLKNKKLAAKDRREESLLPPYYVAGLPGSFYTRLFPDHCVHLFHSSYCLMWRSKVPDELAGGAVLNEGHMYIWETTPQAVVALYRRQFQEDMSLFLRLRHRELVPGGHMVLAFLGRKKSKDVLRGEVSYTWGLLAQALQSLVKQGRVKKDKLDSFNLPFYAPSMDEVRDVITRSQAFDITHIQLFESNWDPHDDDDVEMKMEEDVAAVQSGVNVARSIRAVIGPLIARHFGEHILDDLFELHAKNVAVHLQKVKTKYPVIVVSLQAK

SEQ ID NO: 55 - protein, 229 aa, Saccharomyces cerevisiae
MGGILKNPLALSPEQLAQQDPETLEEFRRQVYENTQKNAKLTSHKRNIPGLDNTKEEGEIIGTSSTFLPKDTLSLKHEQDMLAKMTPEERVQWNQRNLAENEITKKQFQDIHIDEPKTPYQGAVDPHGEYYRVDDDEDEDNSDKKPCQVANDDIDDLSLGEPEFEIKENKQPDFETNDENDEDSPEARHKKFEEMRKKHYDVRAIFNKKSREALKDEDEDEDDSTTKEP

SEQ ID NO: 56 - protein, 71 aa, Synechocystis PCC6803
MFKPSLLLSLLVVGCLLTVTGCGGGNQREEGQPTQQNIEQQENNNGNANTNDDDNDNNDDDEEDDNKDGDD

SEQ ID NO: 57 - protein, 373 aa, Glycine max
MSAVESAEHNNIRGVPTHGGRYVQYNIYGNLFEVSRKYVPPIRPVGRGAYGIVCAAVNAETGEEVAIKKIGNAFDNRIDAKRTLREIKLLRHMDHANIMSIKDIIRPPQKENFNDVYLVSELMDTDLHQIIRSNQQLTDDHCRYFLYQLLRGLKYVHSANVLHRDLKPSNLLLNANCDLKIADFGLARTTSETDFMTEYVVTRWYRAPELLLNCSEYTAAIDIWSVGCILGEIITRQPLFPGKDYVHQLRLITELIGSPDDASLGFLRSDNARRYVKQLPQYPKQNFSARFPTMSPGAVDLLEKMLIFDPNRRITVDEALSHPYMSPLHDINEEPVCTRPFSFDFEQPSFTEEDIKELIWRESVKFNPATIYV

SEQ ID NO: 58 - protein, 539 aa, Saccharomyces cerevisiae
MPLNEKYERPPQPPPAYDPNHRPPSSSENSAAANVNDGQTPYHFRQDQYYNLNSKTSGAPIGSFDEAFPTENDNKPRWNDWPFTIFFLCTVGGFIAIAAITLRAWSQTYSSTGSGIYDGVNTGTLNTNAAILLVFVCIIALVFSVLGLTLCRIFPKQFIYCGMVINLVASLGTAIMYMSLRYWSAGIVFLVFTFMTAWCYWGMRSRIPLSVAVLKVVVDAMKKCPQIFFVSFVGALVASAFGFLFSAVIVATYIKYDPNSSNGGCDVSGGSCSHSKLIGVLVVVFFCGYYISEVIRNVIHCVISGVFGSWYYMSKSDQGMPRWPAFGALKRAMTYSFGSICFGSLLVALIDLLRQILQMIRHDVTSSGGGQIAIQILFMVFDWIIGFLKWLAEYFNHYAYSFIALYGKPYLRAAKETWYMLREKGMDALINDNLINIALGLFSMFASYMTALFTFLYLRFTSPQYNSNGAYNGALMAFSFVIALQICNIATEAIRSGTATFFVALGNDPEVFHHSYPHRFDEIFRAYPDVLRKLSHQNV

SEQ ID NO: 59 - protein, 333 aa, Triticum sp.
MAAQNNKEVDALVEKITGLHAAIAKLPSLSPSPAVDALFTELVTACVPPSPVDVTKLGPEAQEMREGLIRLCSEAEGKLEAHYSDMLAAFDNPLDHLGMFPYYSNYINLSKLEYELLARYVPGGIAPARVAFIGSGPLPFSSFVLAARHLPDTMFDNYDLCGTANDRASKLFRADTDVGARMSFHTADVADLAGELAKYDVVFLAALVGMAAEDKAKVIAHLGAHMADGAALVVRSAHGARGFLYPIVDPQDITLGGFEVLAVCHPDDDVVNSVIIAQKSKDVHVSGLHSGRAVGGQSARGTVPVVSPPCRFGEMVAEVTQKREEFANAEVAF

SEQ ID NO: 60 - protein, 334 aa, Zea mays
MAGAQESLSLVGTMRGHNGEVTAIATPIDNSPFIVSSSRDKSLLVWDLTNPVHSTPESGATADYGVPFRRLTGHSHFVQDVVLSSDGQFALSGSWDGELRLWDLSTGLTTRRFVGHEKDVLSVAFSVDNRQIVSASRDKTIKLWNTLGECKYTIGGDLGGGEGHNGWVSCVRFSPNTFQPTIVSGSWDRTVKVWNLTNCKLRCTLDGHGGYVNAVAVSPDGSLCASGGKDGVTLLWDLAEGKRLYSLDAGSIIHSLCFSPNRYWLCAATQDSVKIWDLESKHVVQDLKPDIQISKNQILYCTSLSWSADGSTLYTGYTDGSIRVWKISGYGYAG

SEQ ID NO: 61 - protein, 201 aa, Saccharomyces cerevisiae
MSSAIVAKLNKEDIIKDTVKDLAFEILGELSVSYVDSDDIKLGNPMPMEATQAAPTIKFTPFDKSQLSAEDKLALLMTDPDAPSRTEHKWSEVCHYIITDIPVEYGPGGDIAISGKGVVRNNYIGPGPPKNSGYHRYVFFLCKQPKGADSSTFTKVENIISWGYGTPGAGAYDYIKENNLQLVGANYYMVENTTVDFNHDM

SEQ ID NO: 62 - protein, 122 aa, Oryza sativa
MAGVWVFKDGIVRRVENPGSEESSSAGDGGGGGRRKVLVHVPSGEVVASYEVLERRLRELGWERYLTDPCLLQFHQRSTVHLISVPRDFSKFKLVHMYDIVVKTRNVFEVRDAAAPAVSPAT

SEQ ID NO: 63 - protein, 118 aa, Oryza sativa
MAGVWVFEDGMVRRADSEAPSRGRGVGGGGGGGKVLVHVPSSEVVTSYEVLERRLRELGWERYLNDPCLLQFHQRSTVHLISVPRDFSRLKLVHMYDVVVKTRNVFEVRDAATTASPP

SEQ ID NO: 64 - protein, 319 aa, Zea mays
MAIKRTKAEKKIAYDKKLCSLLDEYTKVLIALADNVGSKQLQDIRRGLRGDSVVLMGKNTLIRRCIKVYAEKTGNHTFDPLMDLLVGNVGLIFTKGDLKEVREEVAKYKVGAPARVGLVAPVDVVVPPGNTGLDPSQTSFFQVLNIPTKINKGTVEIITPVELIKKGEKVGSSESALLAKLGIRPFSYGLQVTSVYEDGSVFSPEVLDLSEEDLIEKFATGVSMVASLSLAISYPTLAAVPHMFINGYKNVLAVAVETDYSYPHADKIKEYLKDPSKFAVAAPVAAGDSGAAAAPKEEEKAAEPEEESDEEMGFSLFDD

SEQ ID NO: 65 - protein, 348 aa, Zea mays
MENGGAKGDVPDDANEHCPGTQSEEAGKADACAGCPNQQICATAPKGPDPDVVAIVERLATVKHKLLVLSGKGGVGKSTFSAQLSFALAEMDHQVGLLDIDICGPSIPKMLGLEGQDIHQSNLGWSPVYVESNLGVMSIGFMLPNPDDAVIWRGPRKNGLIKQFLKDVDWGEIDYLVVDAPPGTSDEHISIVQYLQATGIDGAIIVTTPQQVSLIDVRKEINFCKKVGVPVLGVVENMSGLRQPLSDLRFIKSDEAGKTDATEWALNYIKEKAPELLSVMACSEVFDSSKGGAEKMCHEMGVPFLGKVPMDPQLCKAAEEGRSCFADQKCSASAPALRNIVKKLIKAE

SEQ ID NO: 66 - protein, 196 aa, Arabidopsis thaliana
MGRRKIEIKRIENKSSRQVTFSKRRNGLIDKARQLSILCESSVAVVVVSASGKLYDSSSGDDISKIIDRYEIQHADELRALDLEEKIQNYLPHKELLETVQSKLEEPNVDNVSVDSLISLEEQLETALSVSRARKAELMMEYIESLKEKEKLLREENQVLASQMGKNTLLATDDERGMFPGSSSGNKIPETLPLLN

SEQ ID NO: 67 - protein, 324 aa, Glycine max
MERSGGMVTGSHERNELVRVRHGSDSRSKPLKNLNGQSCQICGDTIGLTATGDVFVACHECGFPLCHSCYEYELKHMSQSCPQCKTAFTSHQEGAEVEGDDDDEDDADDLDNEINYGQGNSSKAGMLWEEDADLSSSSGHDSQIPNPHLANGQPMSGEFPCATSDAQSMQTTSIGQSEKVHSLSYADPKQPGPESDEEIRRVPEIGGESAGTSASQPDAGSNAGTERVQGTGEGQKKRGRSPADKESKRLKRLLRNRVSAQQARERKKAYLIDLETRVKDLEKKNSELKERLSTLQNENQMLRQILKNTTASRRGSNNGTNNAE

SEQ ID NO: 68 - protein, 110 aa, Zea mays
MAATKLQALWNHPAGPKTIHFWAPTFKWGISIANIADFAKPPEKISYPQQVAVACTGLIWSRYSLVITPRNWNLFSVNVAMAGTGLYQLSRKIRQDYLSDEKDAASQLEA

SEQ ID NO: 69 - protein, 102 aa, Oryza sativa
MRPTGRRSSPPVAAALALLLLLVLFFFSHCASAARPLPASAAAELVLQDGATGNGDEVSELMGAAEEEAAGLCEEGNEECVERRMLRDAHLDYIYTQKRNRP

SEQ ID NO: 70 - protein, 167 aa, Saccharomyces cerevisiae
MSTIPSEIINWTILNEIISMDDDDSDFSKGLIIQFIDQAQTTFAQMQRQLDGEKNLTELDNLGHFLKGSSAALGLQRIAWVCERIQNLGRKMEHFFPNKTELVNTLSDKSIINGINIDEDDEEIKIQVDDKDENSIYLILIAKALNQSRLEFKLARIELSKYYNTNL

SEQ ID NO: 71 - protein, 351 aa, Glycine max
MGRWVNCRGNIFPFLGMVMAMLAQSGSMVVIKVAMTDGINKYVMVVYSLALSTILLLPFALFLHRSERPPLTFSALCSFFLLAFFGSSGQIMAYVGIDLSSPTLASAMLNLIPAFTFILALIFRMEEVHWKHSSSQAKFLGTIVSIAGAFVVILYKGPPIFKTHLSNSSNKFLFSQQLNWILGGMFCAGDSIVCSLWYIYQASVAYKYPAVTTIVFFQLLFSTIQCGVFALIVVQDQTEWELKLDIGLIGIVYQAIAATLIRYILCTWCVLKAGPLFCSMFKPVAIIFSVFMGAIFLGDDLSLGSLIGAVIIVIGFYAVLWGKSIEDNKIEKGVENLESSCHNVPLLQNRT

SEQ ID NO: 72 - protein, 348 aa, Saccharomyces cerevisiae
MSKTAVKDSATEKTKLSESEQHYFNSYDHYGIHEEMLQDTVRTLSYRNAIIQNKDLFKDKIVLDVGCGTGILSMFAAKHGAKHVIGVDMSSIIEMAKELVELNGFSDKITLLRGKLEDVHLPFPKVDIIISEWMGYFLLYESMMDTVLYARDHYLVEGGLIFPDKCSIHLAGLEDSQYKDEKLNYWQDVYGFDYSPFVPLVLHEPIVDTVERNNVNTTSDKLIEFDLNTVKISDLAFKSNFKLTAKRQDMINGIVTWFDIVFPAPKGKRPVEFSTGPHAPYTHWKQTIFYFPDDLDAETGDTIEGELVCSPNEKNNRDLNIKISYKFESNGIDGNSRSRKNEGSYLMH

SEQ ID NO: 73 - protein, 573 aa, Zea mays
MAYFPDEVVGYILGYVTSHQDRNAVSLVCRAWYDIERHGRHSVLVRNCYAVCPERVHMRFPNMRALSLKGKPHFAEFNLVPAGWGATANPWVDACARACPGLEELRLKFMVVTDECLKLLSLSFTNFKSLVLVCCEGFSTTGLANIATNCRFLKELDLQKSCVKHQDHQWINCFPKSSTSLECLNFSCLTGEVNAVALEELVARSPNLKSLRLNLAVPFDVLSRILSRTPKLEDLGTGSFLQGNDPAAYASLCRALENCTSLKSISGFWDAPGFYVQGILSNCKIRNLTCLNLSYATLIQSTQLIGIIRHCKKLHVLWVLDHIGDEGLKAVSFSCPDLQELRVYPSVVAPRGTVTEEGLVALSSCRKLQRVLFSCVRMTNTALMTIARYCPRLTSFRLCIRKPRSADAVTGQPLDEGFGAIVRSCRGLRRLAMSGLLTDSVFLYIGMYAEKLEMLSVTFAGDTDDGMVYVLNGCRNLKKLVIKESPFGDAALLAGAHRYESMRSLWMSSCQITLGGCKALAATMPNINVEVIGGASFGAMDGGVSGARKVDMLYLYRTLAGPRCDTPGFVSIL

SEQ ID NO: 74 - protein, 137 aa, Saccharomyces cerevisiae
MNDGQFLFQRNDPIILYTFLLKSNYIVFRSIDERLCDFVFYIDHFLNKRISYRIPILIRNNNTNILNNCPSSFPPLVDLVGHRLVAAEDNPVAVDLVDNNLVVVDLVDNNLAVGVLVGSNLVVGSLVFALLTCFEDG

SEQ ID NO: 75 - protein, 241 aa, Zea mays
MASCGAGDAASAQPRASISHVIFDMDGLLLDTEGFYTEVQEKILARYDKVFDWSLKAKMMGKKAAESARIFVDECGLNGLLTPEQFLEERESMLQALFPSCTKLPGVLRLVHHLHANGVPMAVATGSHKRHFALKTQNHQEMFSLMHHVVMGDDPEVKAGKPSPDIFLAAMRRFEGGVEPSKCLVFEDAPSGVAAAKNAGMSVVMVPDPRLDVSYHKGADQVLSSLLDFKPAEWGLPAFKE

SEQ ID NO: 76 - protein, 276 aa, Zea mays
MEAVARSGDYLWRFVAETEWYNEVVLSAVAPGDWWRGLPHPVQSWMRNCVGGYLLYFISGFLWCFVIYYWKRHAYIPKDAIPTNEAMKKQIVVASKAMPFYCALPTLSEYMIESGWTRCYFNISEIGFSMYLCYMAMYLIFVEFGIYWMHRELHDIKPLYKYLHATHHIYNKENTLSPFAGLAFHPLDGILQAIPHVFALFLFPTHFRTHIALLFLEAVWTTNIHDCIHGKIWPVMGAGYHTIHHTTYRHNYGHYTIWMDWMFGTLNEPEDILKKA

(19) United States
(12) Patent Application Publication
(10) Pub. No.: US 2008/0092255 A1
(43) Pub. Date: Apr. 17, 2008

(54) Methods of producing and using cold temperature tolerant plants, seeds, and crops

(76) Inventors: Michael D. Edgerton, St. Louis, MO (US); Banu Gopalan, Cleveland, OH (US); Vincent Jung, Belmont, MA (US); Mahesh J. Kulkarni, Pune (IN); Linda L. Madson, Willimantic, CT (US); Manchikanti Padmavathi, West Bengal (IN); Padmini Sudarshana, Bangalore (IN); Geetha Vasanthakumar, Atlanta, GA (US); John Xu, O'Fallon, MO (US); Yihou Zhou, Ballwin, MO (US); Li Zhou, Belmont, MA (US); Kimberly F. Zobrist Duff, St. Louis, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 11/728,976

(22) Filed: Mar. 27, 2007

Related U.S. Application Data
(60) Provisional application No. 60/786,346, filed on Mar. 27, 2006.

(51) Int. Cl. A01H 5/00; A01H 1/00; A01H 5/10; C12N 5/10

(52) U.S. Cl. 800/289; 435/412; 435/415; 435/419; 800/295; 800/298; 800/300; 800/312; 800/314; 800/320.1; 800/320.2; 800/320.3

(57) ABSTRACT

The present disclosure provides transgenic seeds and plants with enhanced cold tolerance and cold vigor. The disclosure also provides methods of producing these transgenic plants and seeds. The present disclosure further provides methods of producing crops that can be grown under sub-optimum conditions and methods of increasing the yield of crop plants by extending growing season of a plant by earlier planting of transgenic seeds of the invention under sub-optimum growth conditions.

This application claims the benefit of U.S. application No. 60/786,346 filed Mar. 27, 2006 hereby incorporated by reference in its entirety.

### INCORPORATION OF SEQUENCE LISTING

Two copies of the sequence listing (Seq. Listing Copy 1 and Seq. Listing Copy 2) and a computer-readable form of the sequence listing, all on CD-ROMS, each containing the file named Cold_multigeneRegularST25.txt, which is 151,552 bytes (measured in MS-DOS) and was created on Mar. 27, 2007, and are herein incorporated by reference.

### FIELD OF THE INVENTION

The invention relates to the field of plant molecular biology and plant genetic engineering. In one aspect, the invention relates to transgenic seeds and plants with improved cold tolerance and methods for making and using the seeds and plants.

### BACKGROUND OF THE INVENTION

Recent advances in genetic engineering have provided the prerequisite tools to transform plants to contain foreign (often referred to as “heterogenous or heterologous”) or improved endogenous genes. The introduction of such a gene in a plant may desirably lead to an improvement of an already existing pathway in plant tissues or introduction of a novel pathway to modify product levels, increase metabolic efficiency, and/or save on energy costs to the cell. It is presently possible to produce plants with unique physiological and biochemical traits and characteristics of high agronomic importance. Traits that play an essential role in plant growth and development as well as crop yield potential, stability, crop quality, and composition are desirable targets for crop plant improvement. These improvements may be achieved by genetically modifying a crop plant for improved stress tolerance.

Any process that can alter the growing season of a crop plant can also alter yields of a desired plant product. One way of achieving this goal is by providing seeds that can germinate earlier in the growing season. Normally, colder temperatures precede the growing season, therefore seeds that can germinate under lower temperatures, for example those that can produce seedlings that are tolerant of lower temperatures, may result in crops with higher yields. We have discovered that transgenic seeds expressing at least one of the functional genes disclosed herein can provide some of these properties to plants.

### OBJECTS OF THE INVENTION

It is an object of the present disclosure to provide transgenic seeds and plants with enhanced cold tolerance by expressing a functional polypeptide encoded by a gene described in SEQ ID NO: 1 through SEQ ID NO: 38 or genes homologous to those described in SEQ ID NO: 1 through SEQ ID NO: 38. Another object of the present disclosure is to provide methods of making transgenic seeds and plants with enhanced cold vigor and cold tolerance by transforming desired plant cells to express a functional polypeptide encoded by at least one of the genes described in SEQ ID NO: 1 through SEQ ID NO: 38 or genes homologous to those described in SEQ ID NO: 1 through SEQ ID NO: 38.

The present disclosure also provides transgenic seeds and plants showing enhanced germination at low temperatures by use of the genes described in SEQ ID NO: 1 through SEQ ID NO: 38 or genes homologous to those described in SEQ ID NO: 1 through SEQ ID NO: 38.

Another object of the present disclosure is to provide transgenic seeds expressing a gene described in SEQ ID NO: 1 through SEQ ID NO: 38 or genes homologous to those described in SEQ ID NO: 1 through SEQ ID NO: 38 with a seed coating permitting the seed to germinate at low temperatures.

Another object of the present disclosure is to provide methods of making hybrid seeds that are capable of expressing at least one of the polypeptides described in SEQ ID NO: 39 through SEQ ID NO: 76 or polypeptides homologous to those described in SEQ ID NO: 39 through SEQ ID NO: 76. The present disclosure also provides methods of making hybrid seeds that are resistant to a variety of herbicides and have enhanced cold tolerance and seedling vigor under cold conditions, and hybrid seeds that are resistant to a variety of insects and have enhanced cold tolerance and seedling vigor under cold conditions. These hybrid seeds may be planted to produce transgenic crops that have enhanced cold tolerance and cold vigor.

Another object of the present disclosure is to provide methods of producing transgenic crops and methods of extending the growing season of crop plants by earlier planting of transgenic seeds expressing a functional polypeptide encoded by any one of the polynucleotides described herein.

The present disclosure also provides plants, seeds, plant parts, and plant products produced by various methods of the invention.

### SUMMARY OF THE INVENTION

We have found that certain transgenic seeds that express a polypeptide encoded by a polynucleotide described herein or its homolog can be selected to have a better ability to germinate under cold conditions, and this property of the transgenic plant can be exploited to extend the growing season of crop plants.

We have discovered that the ectopic expression of polynucleotides encoding polypeptides described herein during seed germination can impart significant tolerance to cold temperatures in selected transgenic plants. The transgenic plants that are selected for enhanced seedling and/or germination vigor may be planted earlier in the season, thereby extending the growing season and increasing the yield of a crop.

In accordance with one aspect of the invention, transgenic plants and seeds of the desired species are transformed with a DNA construct that is capable of expressing a functional polypeptide described in SEQ ID NO: 39 through SEQ ID NO: 76 or polypeptides homologous to those described in SEQ ID NO: 39 through SEQ ID NO: 76 at least during the period of seed germination and early seedling growth. In accordance with another aspect of the invention, transgenic plants and seeds of the desired species are transformed with a DNA construct that is capable of expressing a polypeptide described in SEQ ID NO: 39 through SEQ ID NO: 76 or a polypeptide homologous to those described in SEQ ID NO: 39 through SEQ ID NO: 76 when the seed is germinated in a field under sub-optimal growth conditions, thereby providing a seedling with enhanced cold tolerance.

In accordance with another aspect of the invention, transgenic plants and seeds may be transformed with a DNA construct capable of expressing a functional polypeptide with at least 75% identity to any one of the polypeptides selected from a group consisting of SEQ ID NO: 39 to SEQ ID NO: 76 and may be characterized by a germination index value in a range of 48 to 150 at a temperature of 9.0° C. to 9.8° C. Such transgenic seeds may have a percent germination of seeds greater than 80% at a temperature of 8.0° C. to 9.3° C.

In accordance with another aspect of the invention, transgenic plants and seeds may be comprised of a plurality of plant cells in which recombinant DNA comprising a promoter that is functional in plant cells at least during the period of seed germination and early seedling growth and a polynucleotide sequence described in SEQ ID NO: 1 through SEQ ID NO: 38 or genes homologous to those described in SEQ ID NO: 1 through SEQ ID NO: 38 is stably integrated.

In accordance with another aspect of the invention, transgenic plants and seeds may be produced by a method in which a plant cell is transformed with a DNA construct comprising a promoter that is functional in plant cells at least during the period of seed germination and early seedling growth and a polynucleotide sequence described in SEQ ID NO: 1 through SEQ ID NO: 38 or genes homologous to those described in SEQ ID NO: 1 through SEQ ID NO: 38 and regenerated into a transgenic plant that produces seeds. The seeds may then be screened for enhanced cold tolerance and seeds with enhanced cold tolerance may be selected.

In accordance with another aspect of the invention, transgenic plant seeds may be coated with a seed coating that allows the seed to germinate under suboptimal germination temperatures and additionally may provide insect, bacterial, or fungal resistance to the transgenic seed. Transgenic seeds may also be coated with a hormone, nutrient, herbicide, color, microbial inoculum, avian repellent, rodent repellent, or a combination thereof. Transgenic seeds of the present invention may also be coated with seed coating that increases the flow of seeds for planting or preventing mechanical damage during the planting of seeds.

In accordance with another aspect of the invention, transgenic seeds with enhanced cold vigor and/or cold tolerance are those for which the average temperature of germination is at least about two degrees Celsius less than the average temperature of germination for a non-transformed seed of same plant species.

The invention also relates to hybrid seeds of crop plants, wherein seeds of the present invention are grown to mature plants and crossed with another plant of compatible species to produce hybrid seeds that are tolerant to cold temperatures.

In accordance with another aspect of the invention, a method of producing a crop, for example a crop with increased yield, is provided. The method comprises planting a transgenic seed of the invention and growing the transgenic plant to obtain a crop or a terminal crop, whereby yield of the transgenic plant is increased as compared to non-transgenic plant of similar genotype.

In accordance with another aspect of the invention, a method of increasing root and shoot biomass of a crop or terminal crop by planting transgenic seeds of the invention is provided. The method comprises planting a transgenic seed of a plant with a DNA construct expressing a polynucleotide molecule encoding a functional polypeptide described in SEQ ID NO: 39 through SEQ ID NO: 76 or a polypeptide homologous to those described in SEQ ID NO: 39 through SEQ ID NO: 76 that is selected for enhanced cold tolerance and growing the plant from the seed to obtain a crop or terminal crop, whereby the root and shoot biomass of the transgenic plant seedling is increased as compared to a non-transgenic plant seedling of similar genotype.

In accordance with another aspect of the invention, a method of extending a growing season of a plant is provided. The method comprises planting a transgenic seed of a plant with a DNA construct expressing a polynucleotide molecule encoding a functional polypeptide of this invention that is selected for enhanced seedling/germination vigor. This aspect of the invention also provides transgenic seed for growing a transgenic plant that has enhanced tolerance to cold temperatures. The genome of such a transgenic plant will comprise a recombinant DNA construct that expresses a functional polypeptide described in SEQ ID NO: 39 through SEQ ID NO: 76 or a polypeptide homologous to those described in SEQ ID NO: 39 through SEQ ID NO: 76.

This invention relates to plants or plant organs produced from the transgenic seed of the present invention. Transformed plants selected for cold temperature tolerance should provide seeds with faster germination under cold conditions and faster emergence under cold conditions, leading to better plant stand, increased growth rates, greener and/or larger leaves and other vegetative parts, increased root mass, and increased biomass of the plant as compared to non-transgenic plants of similar genotype that are retarded by or succumb to cold temperatures.

### BRIEF DESCRIPTION OF THE FIGURES

FIG. 1 shows a plasmid map for a plant transformation vector PMON 75303.

### DETAILED DESCRIPTION OF THE INVENTION

We have found that transgenic plant seeds expressing polypeptides disclosed herein or their homologs have a better ability to germinate under cold conditions. This property of the transgenic plant seeds can be exploited to extend the growing season of crop plants and effectively increase yield of a crop or terminal crop. According to the present disclosure, “yield” is defined as a product or by-product obtained from a plant as a result of cultivation.

We have also discovered that ectopic expression of at least one of the polynucleotide molecules disclosed herein during seed germination can impart a significant advantage to germination at cold temperatures. These transgenic plants can provide seeds that can be planted earlier in the season, thus extending the growing season, and can also result in a higher yield of a desired crop or a terminal crop. The polynucleotide molecules that may be expressed in these transgenic plants are identified as SEQ ID NO: 1 to SEQ ID NO: 38 and encode the polypeptide identified as SEQ ID NO: 39 to SEQ ID NO: 76.

In accordance with an aspect of the invention, the seed according to the invention can be planted, grown, and harvested to produce a crop or terminal crop. As used herein, a “crop” is a plant or plant product that is grown and harvested, such plant or plant product including but not limited to plants or plant parts such as leaf, root, shoot, fruit, seed, grain, or the like. A “terminal crop” is a crop grown for uses other than for use as planting seed to produce subsequent generations of plants. In some crop plants, such as grain produced from hybrid corn, the crop is not very suitable for planting because it does not breed true and the crop can then be conveniently referred to as hybrid grain. In other crop plants, where the crop does breed true, such as soybean, whether a crop is planting seed or a terminal crop will depend on the uses and marketing channels of the crop. If used or marketed for planting, it will be a crop of planting seed; if used or marketed for other purposes it will be a terminal crop.

In accordance with yet a further aspect of the invention, the invention comprises by-products produced from plant or plant product produced from seed in accordance with the invention. A plant by-product includes any product that is made from a plant or plant product, for example, by dehulling, crushing, milling, extraction, hydrogenation, and other processes. A plant by-product in accordance with the present disclosure therefore, will include, for example, dehulled soybeans, crushed corn, soybean meal, soy milk, paper made from corn stalks, and a wide range of other useful products of processing based on plant vitamins, minerals, lipids, proteins, and carbohydrates, and their constituents that can be characterized as being produced from crops or terminal crops in accordance with the invention.

One aspect of the invention relates to polynucleotide molecules, disclosed herein, capable of allowing transgenic seeds to germinate under cold conditions when these molecules are transformed in a plant to produce seeds capable of expressing encoded polypeptide molecules. In vivo ectopic expression of these polynucleotide molecules also helps to protect early seedlings from cold damage, thus improving cold stress tolerance of the plant. It is desirable to enhance cold tolerance in crop plants that undergo such a stress over the course of a normal growing season. The capability of withstanding stress by plants is directly related to the plants' overall general health and is referred to as plant vigor in the art. As used herein, “plant vigor” is defined as the capacity for natural growth and survival. Components of plant vigor include, but are not limited to, faster germination, faster emergence, better plant stand, increased growth rates, greener or larger leaves and other vegetative parts, increased root mass, and increased biomass of the plant. According to the present invention, “enhanced germination vigor” is the vigor of a seed that will result in faster germination and faster emergence under optimum or sub-optimum conditions leading to a plant with enhanced vigor as compared to an un-enhanced seed. Seeds, seedlings, and plants with cold temperature vigor will have enhanced capacity for natural growth and survival under colder conditions encountered in a green house, growth chamber or in a field as compared to seeds, seedlings, and plants without such vigor. Seed vigor, seedling vigor, and plant vigor can be determined by performing a variety of tests either individually or in combination with other tests. Examples of tests performed for determination of vigor in optimum or sub-optimum conditions include but are not limited to germination assay, germination index or percent germination determination, growth assays such as early seedling growth assay, and different kinds of shock assays such as cold shock assay.

Homologs are expressed by homologous genes, which are genes that encode proteins with the same or similar biological function. Homologous genes may be generated by the event of speciation (ortholog) or by the event of genetic duplication (paralog). Orthologs refer to a set of homologous genes in different species that evolved from a common ancestral gene by specification. Normally, orthologs retain the same function in the course of evolution. Paralogs refer to a set of homologous genes in the same species that have diverged from each other as a consequence of genetic duplication. Thus, homologous genes can be from the same or a different organism. Homologous DNA includes naturally-occurring and synthetic variants. For instance, degeneracy of the genetic code provides the possibility of substituting at least one base of the protein-encoding sequence of a gene with a different base without causing the amino acid sequence of the polypeptide produced from the gene to be changed. Hence, a polynucleotide useful in the present invention may have any base sequence in SEQ ID NO: 1 through SEQ ID NO: 38 changed by substitution in accordance with degeneracy of the genetic code. Genes that are substantially homologous to those in SEQ ID NO: 1 through SEQ ID NO: 38 will have at least 60% identity, at least 70%, at least 80%, or at least 90% identity over the full length of genes described in SEQ ID NO: 1 through SEQ ID NO: 38. Substantially homologous genes encode proteins that, when optimally aligned, have at least 60% identity, at least 70%, at least 80%, or at least 90% identity over the full length of a protein described in SEQ ID NO: 39 through SEQ ID NO: 76, or a higher percent identity over a shorter functional part of the protein, e.g., at least 70%, at least 80%, or at least 90% amino acid identity over a window of comparison comprising a functional part of the protein imparting the enhanced agronomic trait. Polypeptides that are substantially homologous to those in SEQ ID NO: 39 through SEQ ID NO: 76 will have at least 60% identity, at least 70%, at least 80%, or at least 90% identity over the full length of proteins described in SEQ ID NO: 39 through SEQ ID NO: 76, or a higher percent identity over a shorter functional part of the protein, e.g. at least 70%, at least 80%, or at least 90% amino acid identity over a window of comparison comprising a functional part of the protein imparting the enhanced agronomic trait.

Homologs can be identified by comparison of amino acid sequence, e.g. manually or by using known homology-based search algorithms such as those commonly known and referred to as BLAST, FASTA, and Smith-Waterman. A local sequence alignment program, e.g. BLAST, can be used to search a database of sequences to find similar sequences, and the summary Expectation value (E-value) may be used to measure the sequence base similarity. As a protein hit with the best E-value for a particular organism may not necessarily be an ortholog or the only ortholog, a reciprocal query is used in the present invention to filter hit sequences with significant E-values for ortholog identification. The reciprocal query entails a search of the significant hits against a database of amino acid sequences from the base organism that are similar to the sequence of the query protein. A hit is a likely ortholog when the reciprocal query's best hit is the query protein itself or a protein encoded by a duplicated gene after speciation.

A further aspect of the invention comprises functional homolog proteins that differ in one or more amino acids from those of the disclosed protein as the result of conservative amino acid substitutions, e.g. substitutions that are among: acidic (negatively charged) amino acids such as aspartic acid and glutamic acid; basic (positively charged) amino acids such as arginine, histidine, and lysine; neutral polar amino acids such as glycine, serine, threonine, cysteine, tyrosine, asparagine, and glutamine; neutral nonpolar (hydrophobic) amino acids such as alanine, leucine, isoleucine, valine, proline, phenylalanine, tryptophan, and methionine; amino acids having aliphatic side chains such as glycine, alanine, valine, leucine, and isoleucine; amino acids having aliphatic-hydroxyl side chains such as serine and threonine; amino acids having amide-containing side chains such as asparagine and glutamine; amino acids having aromatic side chains such as phenylalanine, tyrosine, and tryptophan; amino acids having basic side chains such as lysine, arginine, and histidine; amino acids having sulfur-containing side chains such as cysteine and methionine; naturally conservative amino acids such as valine-leucine, valine-isoleucine, phenylalanine-tyrosine, lysine-arginine, alanine-valine, aspartic acid-glutamic acid, and asparagine-glutamine. A further aspect of the homologs encoded by DNA useful in the transgenic plants of the invention are those proteins that differ from a disclosed protein as the result of deletion or insertion of one or more amino acids in a native sequence.

In order to practice the present invention, it is essential to introduce the selected polynucleotide molecule in a form that is capable of producing active polypeptide molecules in a desired plant. Exogenous polynucleic acid molecules are transferred into a crop plant cell by the use of a recombinant DNA construct (or vector) designed for such a purpose.

A plant recombinant DNA construct of the present invention contains a structural nucleotide sequence encoding a polypeptide of the present invention and operably linked to regulatory sequences. The DNA constructs may be double border plant transformation constructs that also contain DNA segments that provide replication function and antibiotic selection in bacterial cells, for example, an E. coli origin of replication such as ori322, a broad host range origin of replication such as oriV or oriRi, and a coding region for a selectable marker such as Spc/Str that encodes Tn7 aminoglycoside adenyltransferase (aadA) conferring resistance to spectinomycin or streptomycin, or a gentamicin (Gm, Gent) or one of many known selectable marker genes.

These constructs also may contain at least one expression cassette capable of integrating in a plant genome, expressing a functional polypeptide, and providing a means to select transgenic plants expressing polypeptides of the invention. A functional polypeptide of the invention provides a function in a plant comparable to the native protein including but not limited to the natural polypeptides. For example, a cassette may contain a promoter operably linked to an intron and/or desired polynucleotide followed by a transcription termination sequence flanked by T-DNA integration sites isolated from Agrobacterium. Construction of such a vector is well-known to those skilled in the art. The expression cassette used for transforming plants to practice the current invention comprises any one of the known promoters that cause the transcription of the desired gene in plant cells and any one of the known antibiotic or herbicide tolerance-encoding polynucleotide sequences known to confer antibiotic or herbicide tolerance to the plant cells.

In accordance with the present disclosure, “expression” means the transcription and stable accumulation of sense or antisense mRNA or polypeptide derived from the polynucleotide of the present invention in a plant. “Ectopic expression” refers to the expression of an RNA molecule in a cell type other than a cell type in which the RNA is normally expressed, or at a time other than a time at which the RNA is normally expressed, or at an expression level other than the level at which the RNA normally is expressed. The promoter that causes expression of an RNA that is operably linked to the polynucleotide molecule in a construct usually controls the expression pattern of the translated polypeptide in a plant. Promoters for practicing the invention may be obtained from various sources including, but not limited to, plants and plant viruses. Several promoters, including constitutive promoters, inducible promoters, tissue-specific promoters, and tissue-enhanced promoters that are active in plant cells have been described in the literature. For example, a promoter may be selected from those that cause sufficient expression to result in the production of an effective amount of a polypeptide to cause the desired phenotype.

In accordance with the current invention, constitutive promoters are active under most environmental conditions and states of development or cell differentiation. These promoters are likely to provide expression of the polynucleotide sequence at many stages of plant development and in a majority of tissues. A variety of constitutive promoters are known in the art. Examples of constitutive promoters that are active in plant cells include but are not limited to the nopaline synthase (NOS) promoters; the cauliflower mosaic virus (CaMV) 19S and 35S promoters (U.S. Pat. No. 5,858,642); the figwort mosaic virus promoter (P-FMV, U.S. Pat. No. 6,051,753); and actin promoters, such as the rice actin promoter (P-Os.Act1, U.S. Pat. No. 5,641,876).

In another embodiment, the gene of the invention in a DNA construct may be ectopically expressed by using an inducible promoter. Inducible promoters cause conditional expression of a polynucleotide sequence under the influence of changing environmental conditions or developmental conditions. For example, such promoters may cause expression of the polynucleotide sequence at certain temperatures or temperature ranges, or in specific stage(s) of plant development, such as in early germination or in the late maturation stage(s) of a plant. Examples of inducible promoters include, but are not limited to, the light-inducible promoter from the small subunit of ribulose-1,5-bis-phosphate carboxylase (ssRUBISCO); the drought-inducible promoter of maize (Busk et al., Plant J. 11:1285-1295, 1997); the cold, drought, and high salt inducible promoter from potato (Kirch, Plant Mol. Biol. 33:897-909, 1997); and many cold inducible promoters known in the art, for example rd29a and cor15a promoters from Arabidopsis thaliana (Genbank ID: D13044 and U01377), blt101 and blt4.8 from barley (Genbank ID: AJ310994 and U63993), wcs120 from wheat (Genbank ID:AF031235), and mlip15 from corn (Genbank ID: D26563).

For example, a germination-specific promoter may be most highly expressed in the appropriate tissues and cells at the appropriate developmental time to express polynucleotides of the invention only during germination or early seedling growth. Tissues and cells that comprise the germination and early seedling growth stages of plants may include: the radical, hypocotyl, cotyledons, epicotyl, root tip, shoot tip, meristematic cells, seed coat, endosperm, true leaves, internodal tissue, and nodal tissue. Germination-enhanced promoters have been isolated from genes encoding the glyoxysomal enzymes isocitrate lyase (ICL) and malate synthase (MS) from several plant species (Zhang et al, Plant Physiol. 104: 857-864, 1994; Reynolds and Smith, Plant Mol. Biol. 27: 487-497, 1995; Comai et al, Plant Physiol. 98: 53-61, 1992). Other promoters include SIP-seedling imbibition protein promoter (Heck, G., Ph.D. Thesis, 1992, Washington University, St. Louis, Mo.) and cysteine endopeptidase promoter (Yamauchi et al, Plant. Mol. Biol. 30: 321-329, 1996). Additionally, promoters could be isolated from other genes whose mRNAs appear to accumulate specifically during the germination process, for example class I β-1,3-glucanase B from tobacco (Vogeli-Lange et al., Plant J. 5: 273-278, 1994), canola cDNAs CA25, CA8, AX92 (Harada et al., Mol. Gen. Genet. 212: 466-473, 1988; Dietrich et al., J. Plant Nutr. 8: 1061-1073, 1992), lipid transfer protein (Sossountzove et al, Plant Cell 3: 923-933, 1991), rice serine carboxypeptidases (Washio and Ishikawa, Plant Phys. 105: 1275-1280, 1994), and repetitive proline rich cell wall protein genes (Datta and Marcus, Plant Mol. Biol. 14: 285-286, 1990).

Tissue-specific promoters can also be used in an expression cassette of the invention. Tissue-specific promoters cause transcription or enhanced transcription of a polynucleotide sequence in specific cells or tissues at specific times during plant development, such as in vegetative or reproductive tissues. Examples of tissue-specific promoters under developmental control include promoters that initiate transcription primarily in certain tissues, such as vegetative tissues, e.g., roots, leaves or stems, or reproductive tissues, such as fruit, ovules, seeds, pollen, pistils, flowers, or any embryonic tissue, or any combination thereof. Reproductive tissue-specific promoters may be, e.g., ovule-specific, embryo-specific, endosperm-specific, integument-specific, seed coat-specific, pollen-specific, petal-specific, sepal-specific, or some combination thereof. Tissue-specific promoters also include those that can cause transcription or enhanced transcription in a desired plant tissue at a desired plant developmental stage. Examples of such promoters include, but are not limited to, seedling- or early seedling-specific promoters. One skilled in the art will recognize that tissue-specific promoters may drive expression of operably linked polynucleotide molecules in tissues other than the target tissue. Thus, as used herein, a tissue-specific promoter is one that drives expression preferentially not only in the target tissue, but may also lead to some expression in other tissues as well.

In accordance with the present invention, the expression cassette can have a translation leader sequence between the promoter and the coding sequence. The translation leader sequence may be present in the fully processed mRNA upstream of the translation start sequence. The translation leader sequence may affect processing of the primary transcript to mRNA, mRNA stability, or translation efficiency. Examples of translation leader sequences include maize and petunia heat shock protein leaders, plant virus coat protein leaders and plant rubisco gene leaders, among others (Turner and Foster, Molecular Biotechnology 3:225, 1995).

The coding sequences in the expression cassette may be followed by a “3′ non-translated sequences” or “3′ termination region” that includes sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA. An example of a polyadenylation sequence is the nopaline synthase 3′ sequence (nos 3′; Fraley et al., Proc. Natl. Acad. Sci. USA 80: 4803-4807, 1983).

To allow selection of plant or bacterial cells having DNA constructs of the invention, the DNA construct may be designed with a suitable selectable marker that can confer antibiotic or herbicide tolerance to the cell. Antibiotic resistance can be conferred by including an antibiotic tolerance polynucleotide sequence in the construct. Examples of antibiotic tolerance polynucleotide sequences include, but are not limited to, polynucleotide sequences encoding for proteins involved in tolerance to kanamycin, neomycin, hygromycin, and other antibiotics known in the art. Herbicide tolerance can be conferred by including a herbicide tolerance polynucleotide sequence in the construct. Examples of herbicide tolerance polynucleotide sequences include, but are not limited to, those encoding 5-enolpyruvylshikimate-3-phosphate synthase (EPSPS, described in U.S. Pat. Nos. 5,627,061, and 5,633,435, herein incorporated by reference in their entirety; Padgette et al. (1996) Herbicide Resistant Crops, Lewis Publishers, 53-85; and Penaloza-Vazquez, et al. (1995) Plant Cell Reports 14:482-487) and aroA (U.S. Pat. No. 5,094,945) for glyphosate tolerance; bromoxynil nitrilase (Bxn) for Bromoxynil tolerance (U.S. Pat. No. 4,810,648); phytoene desaturase (crtI (Misawa et al. (1993) Plant Journal 4:833-840, and Misawa et al. (1994) Plant Journal 6:481-489) for tolerance to norflurazon; acetohydroxyacid synthase (AHAS, Sathasiivan et al. (1990) Nucl. Acids Res. 18:2188-2193). Herbicides for which transgenic plant tolerance has been demonstrated and for which the method of the present invention can be applied include, but are not limited to: glyphosate herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides, and glufosinate herbicides. In addition to the foregoing herbicides, there are auxin-like herbicides that mimic or act like natural plant growth regulators called auxins. “Auxin-like herbicides” are also called auxinic or growth regulator herbicides or synthetic auxins or Group 4 herbicides (based on their mode of action). The group of auxin-like herbicides includes four chemical families: phenoxy, carboxylic acid (or pyridine), benzoic acid, and the newest family quinaline carboxylic acid. Dicamba(3,6-Dichloro-2-methoxybenzoic acid) is an example of auxin-like herbicide from the benzoic acid family. Other examples of auxin-like herbicides include (2,4-dichlorophenoxy)acetic acid, commonly known as 2,4-D, 4-(2,4-dichlorophenoxy)butyric acid(2,4-DB), 2-(2,4-dichlorophenoxy)propanoic acid (2,4-DP), (2,4,5-trichlorophenoxy)acetic acid (2,4,5-T), 2-(2,4,5-Trichlorophenoxy)Propionic Acid(2,4,5-TP), 2-(2,4-dichloro-3-methylphenoxy)-N-phenylpropanamide(clomeprop), (4-chloro-2-methylphenoxy)acetic acid(MCPA), 4-(4-chloro-o-tolyloxy)butyric acid(MCPB), and 2-(4-chloro-2-methylphenoxy)propanoic acid(MCPP), 3,6-dichloro-2-pyridinecarboxylic acid(Clopyralid), 4-amino-3,5,6-trichloro-2-pyridinecarboxylic acid(picloram), (2,4,5-trichlorophenoxy)acetic acid(triclopyr), and 4-amino-3,5-dichloro-6-fluoro-2-pyridyloxyacetic acid(fluroxypyr), 3-amino-2,5-dichlorobenzoic acid(choramben), 3,7-dichloro-8-quinolinecarboxylic acid(quinclorac), and 7-chloro-3-methyl-8-quinolinecarboxylic acid(quinmerac).

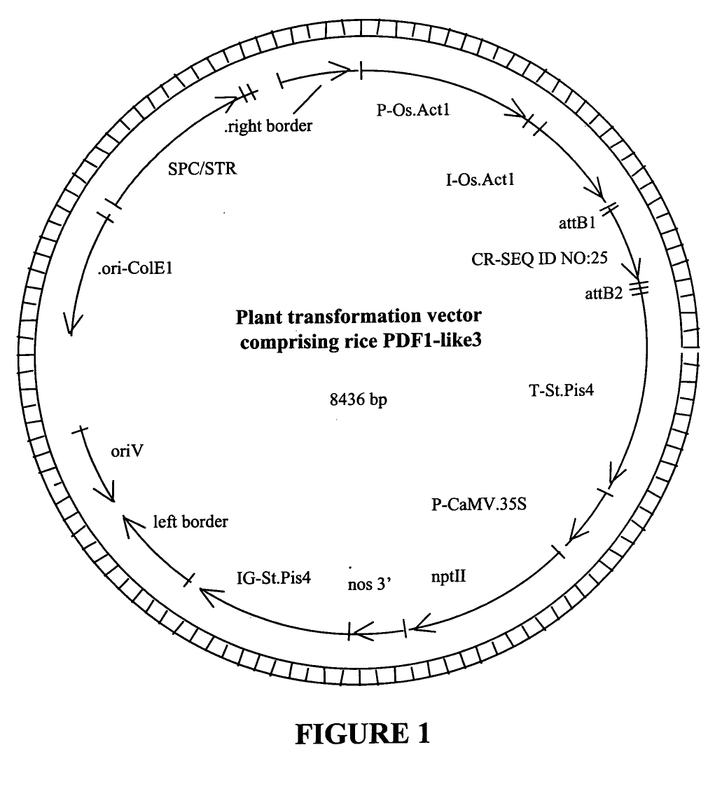

The components of an expression cassette in a DNA construct (expression vector) of the invention may be operably linked with each other in a specific order to cause the expression of the desired gene product in a plant. An example of the order in which components of an expression vector are operably linked is shown in FIG. 1. Right and left borders in this figure flank the expression cassette.

The expression cassette may be assembled in a circular DNA construct, known as a vector backbone, in order to generate isolated desired amounts of DNA in E. coli. Numerous cloning vectors useful in practicing the invention have been described in the literature and some are commercially available. After each cloning, the cloning vector with the desired insert may be isolated and subjected to further manipulation, such as restriction digestion, insertion of new fragments or nucleotides, ligation, deletion, mutation, resection, etc., so as to tailor the components of the desired sequence. Once the construct has been completed, it may then be transferred to an appropriate vector for further manipulation in accordance with the manner of transformation of the host cell.

Transforming desired constructs capable of expressing one or more polypeptides of the present disclosure can produce transgenic plants. Transgenic corn can be produced by particle bombardment transformation methods as described in U.S. Pat. No. 5,424,412. According to this method, the vector DNA is digested with suitable restriction endonucleases to isolate a plant expression cassette that expresses the polypeptides of the present invention in the plant. The desired expression cassette is purified by agarose gel electrophoresis, then bombarded into embryogenic corn tissue culture cells using a Biolistic® (Dupont, Wilmington, Del.) particle gun with purified isolated DNA fragment. Transformed cells are selected by growing them in a selection media. One example of such a selection step where the aroA:CP4 gene is part of expression cassette is the use of glyphosate (N-phosphonomethyl glycine and its salts) in the media. Whole plants are regenerated, then grown under greenhouse conditions. Fertile seed is collected, planted, and screened for a selectable marker; for example plants expressing the desired polypeptide of the invention along with a aroA:CP4 gene product can be screened by spraying glyphosate to select for glyphosate tolerant plants. Plants expressing the desired polypeptide of the invention can then be backcrossed into commercially acceptable corn germplasm by methods known to those skilled in the art of corn breeding (Sprague et al., Corn and Corn Improvement 3^{rd} Edition, Am. Soc. Agron. Publ (1988).

Transgenic corn plants can also be produced by an Agrobacterium-mediated transformation method. A disarmed Agrobacterium strain C58 (ABI) harboring a DNA construct can be used for such transformations. According to this method, the construct is transferred into Agrobacterium by a triparental mating method (Ditta et al., Proc. Natl. Acad. Sci. 77:7347-7351). Liquid cultures of Agrobacterium are initiated from glycerol stocks or from a freshly streaked plate and grown overnight at 26° C.-28° C. with shaking (approximately 150 rpm) to mid-log growth phase in liquid LB medium, pH 7.0, containing 50 mg/l kanamycin, 50 mg/l streptomycin, and spectinomycin, and 25 mg/l chloramphenicol with 200 μM acetosyringone (AS). The Agrobacterium cells are resuspended in the inoculation medium (liquid CM4C) and the density is adjusted to OD₆₆₀ of 1. Freshly isolated Type II immature HiII×LH198 and Hill corn embryos are inoculated with Agrobacterium containing at least one DNA construct disclosed herein and co-cultured 2-3 days in the dark at 23° C. The embryos are then transferred to delay media (N6 1-100-12/micro/Carb 500/20 μM AgNO3) and incubated at 28° C. for 4 to 5 days. All subsequent cultures are kept at this temperature. Coleoptiles are removed one week after inoculation. The embryos are transferred to the first selection medium (N61-0-12/Carb 500/0.5 mM glyphosate). Two weeks later, surviving tissues are transferred to the second selection medium (N61-0-12/Carb 500/1.0 mM glyphosate). Surviving callus is subcultured every 2 weeks until events can be identified. This usually takes 3 subcultures on a desired selection media. Once events are identified, tissue is bulked up for regeneration. For regeneration, callus tissues are transferred to the regeneration medium (MSOD, 0.1 μM ABA) and incubated for two weeks. The regenerating calli are transferred to a high sucrose medium and incubated for two weeks. The plantlets are transferred to MSOD media in a culture vessel and kept for two weeks. Then the plants with roots are transferred into soil.

Soybean transformation is performed essentially as described in WO 00/42207, herein incorporated by reference in its entirety.

After identifying appropriated transformed plants, plants can be grown to produce desired quantities of seeds of the invention.

The transgenic plant seeds of the present invention may have capability of germinating under cold conditions and may provide plants with increased tolerance to cold temperature due to the expression of an exogenous polynucleic acid molecule encoding a polypeptide of the present invention. The transgenic plant seeds of the present invention may have tolerance to thermal stress, for example, variation from optimal to sub-optimal temperature conditions. “Cold,” sometimes referred to as “sub-optimal” temperature, is defined as thermal conditions below those optimal conditions for normal growth of non-transgenic plants of a similar type or variety. Most seed-bearing plants have a life that starts with active vegetative growth, followed by a reproductive stage leading to seed formation. Seeds remain dormant until favorable conditions are resumed, causing the seeds to germinate and produce a plant. Germination is the resumption of active growth of a seed that results in rupture of the seed coat and emergence of a seedling. Germination includes the following physiological and morphological events: (1) imbibition and adsorption of water, (2) hydration of tissue, (3) absorption of oxygen, (4) activation of enzymes, (5) transportation of hydrolyzed molecules to the embryo axis, (6) increase in respiration and assimilation, (7) initiation of cell division and enlargement, and (8) embryo emergence. Except for imbibition, germination involves numerous enzymatically-controlled processes of catabolism and anabolism (metabolism) and hence is highly responsive to temperature. Maximum, optimum, and minimum temperatures (cardinal temperatures) for germination of most crop seeds are essentially those of normal vegetative growth. The optimum temperature is the one giving highest germination percentage in the shortest period of time. Non-after-ripened seeds with partial, or relative, dormancy germinate in a narrow range of temperatures, ranging for example from 5° C. to 15° C. for low temperature species. After-ripened seeds, which are found in the cultivars of most crops and require a process of seed maturation, do not have such a narrow germination temperature range. Cardinal temperatures of different crop seeds overlap, but the germination rate of all is slower at low temperatures. Seeds of some species, such as cotton, are very sensitive to chilling during germination, especially during imbibition. Germination of seeds of many grasses and trees are benefited by diurnal temperature variations. Cardinal temperatures for different plants vary over a wide range. Examples of cardinal temperatures for few plants are shown in Table A.

TABLE A

| | Minimum | | Maximum |
|---|---|---|---|
| Seed | Temp. | Optimum Temp. | Temp. |
| Corn | 8-10° C. | 32-35° C. | 40-45° C. |
| Rice | 10-12° C. | 30-37° C. | 40-42° C. |
| Wheat | 3-5° C. | 15-31° C. | 30-43° C. |
| Barley | 3-5° C. | 19-27° C. | 30-40° C. |
| Rye | 3-5° C. | 25-31° C. | 30-40° C. |
| Oat | 3-5° C. | 25-31° C. | 30-40° C. |
| Buckwheat | 3-5° C. | 25-31° C. | 35-45° C. |
| Field bindweed | 0.5-3° C. | 20-35° C. | 35-40° C. |
| (Convolvulus arvensis) | | | |
| Tobacco (Florida cigar | 10° C. | 24° C. | 30° C. |
| wrapper) | | | |

Temperature ranges for germination of different seeds. Source: Mayer, A. M., and A. Poljakoff-Mayber. 1963. The Germination of Seed. New York. Macmillan

As used herein, “cold germination” refers to germination occurring at temperatures below, for example two or more degrees Celsius below, those normal for a particular species or particular strain of a plant. In one embodiment, a transgenic plant seed of the invention may germinate at a temperature ranging from 0.2° C. to 10° C. below the minimum germination temperature of a similar non-transgenic plant seed. In another embodiment, a transgenic plant seed of the invention may germinate at a temperature ranging from 0.2° C. to 8° C. below the minimum germination temperature of a similar non-transgenic plant seed. In yet another embodiment, a transgenic plant seed of the invention may germinate at a temperature ranging from 0.2° C. to 5° C. below the minimum germination temperature of a similar non-transgenic plant seed. Under these conditions, transgenic seeds of the invention may have a percent germination ranging from 40% to 99.99%. In an embodiment, under these conditions transgenic seeds of the invention may have a percent germination ranging from 60% to 99%. In another embodiment, under these conditions transgenic seeds of the invention may have a percent germination ranging from 80% to 100%.

Where the transgenic seed is a transgenic corn seed expressing any of the disclosed polypeptides, the minimum germination temperature may range from about 8.0° C. to about 9.8° C. In some embodiments, the transgenic corn seeds may have a germination index value ranging from about 48 to about 150, for example from about 50 to about 150, such as from about 52 to about 150, at a temperature ranging from about 9.0° C. to about 9.8° C. In other embodiments, transgenic corn seed may have a percent germination of greater than 50%, for example greater than 60%, such as greater than 70%, for instance greater than 80%, at a temperature ranging from about 9.0° C. to about 9.8° C. In yet other embodiments, seeds may germinate within about 5 to about 25 days, for example from about 5 to about 20 days, such as from about 5 to about 15 days.

As used herein, “cold tolerance” is defined as the ability of a plant to continue growth for a significant period of time after being placed at a temperature below that typically encountered by a plant of that species at that growth stage. The transgenic seeds of the present invention may have higher tolerance to cold, higher germination in cold temperature, and/or a higher yield of agricultural products under cold stress conditions. The transgenic seedling may have enhanced vigor. The earlier planting of a transgenic seed of the present invention when soil temperatures are at suboptimum growth or germination temperatures exposes them to a greater vulnerability to infection.

The transgenic seeds of the present invention and hybrid seeds, made by growing a transgenic seed of the present invention into a plant to the reproductive stage and crossing it with a second plant, may have a protective seed coating. Recent technological innovations in the agriculture industry allow farmers to plant seeds earlier in the season when soil temperatures are below optimum germination temperature of a crop plant. Earlier planting can be achieved by protecting seeds with a polymer seed coating that delays exposure of seed to the soil until the soil reaches the optimum germination temperature. An example of such a polymer seed coating is IntelliCoat® from Landec Labs, Inc. (Menlo Park, Calif.). Temperature-sensitive polymer coating may provide the benefits of earlier planting, better management of the farmer's time and reduced drying cost of seeds, but will not allow to extend the growing season by earlier germination of seeds and preventing cold and other kinds of damage to the seed or seedlings under cold conditions.

Although seed coatings that do not allow seeds to germinate under suboptimum growth or germination temperatures may not be the preferred seed coating for practicing the present invention, they may be used in appropriate cases for practicing the present invention. The desired seed coatings for practicing the present invention will allow the seeds to germinate under suboptimum growth or germination temperatures. The desired seed coating for germination at a selected range of temperatures can be custom-made by vendors (see, e.g., U.S. Pat. No. 5,129,180, assigned to Landec Labs, Inc. Menlo Park, Calif., herein incorporated in its entirety). Coated seeds of the present invention will comprise a DNA construct comprising a polynucleotide molecule expressing a functional polypeptide of the invention during germination and early growth of the plant.

The coated seeds of the present invention may be in a size range that allows them to be efficiently planted with a mechanical planter. The preferred coating will not interfere with the natural respiration of the seed and will not inhibit germination under suboptimum growth or germination temperatures. Furthermore, the preferred coating loses mechanical integrity when wetted, thereby minimizing inhibition of emergence. The preferred coats also permit coated seeds of the invention to be stored for long periods of time under normal storage conditions without adverse effects.

Further, preferred seed coating for the seeds of the present invention provides a convenient vehicle for incorporation of additives with the seed, such as growth stimulants, fertilizers, etc., that are known to impart desirable effects when placed in close proximity to the germinating seed under cold conditions. The additive may be one or more ingredient selected from the class comprising fungicides, insecticides, rhodenticides, herbicides, bird repellants, nematocides, miticides, dyes, disinfectants, and microbial culture or spores. Examples of growth regulators include giberillic acid, auxins, cytokinins, and other plant hormones. Examples of nutrients include potassium-containing salts, nitrate-containing salts, iron-containing salts, magnesium-containing salts, phosphorus-containing salts, and other micronutrients required for plant growth. Nutrients also include fertilizers. Examples of fungicides include Carboxin, Captan, Difenoconazol, Fludioxonil, Metalaxyl, Mefanoxam, Meneoxam, Thiram, Tebuconazole, or other fungicides, which can be used for protecting seeds from fungal infections when they are in the soil or in storage. The insecticidal additive may include neo-nicotinide insecticides such as imidacloprid, acetamprid, and thiametoxam; carbaztes insecticides such as bifenazate; pyrethroid ether insecticides such as etofenprox and flufenprox; and pyridine azomethine insecticides such as pymetrozine.

Seeds of the present invention may also have a seed coating with inoculums of beneficial microorganisms. Inoculums may be in the form of living cells, lyophilized cells or spores. Beneficial organisms of such seed coating can be selected from Rhizobium, Bradyrhizobium, Pseudomonas, Serratia, Bacillus, Pasteuria, Azotobacter, Enterobacter, Azospirillum, Cynobacteria, Gliocldium, Trichoderma, Coniotherium, Verticillium, Paecilomyces, Metarhizium, Mycorrhizal fungi and Entomophilic nematodes.

Plants of the present invention include, but are not limited to, acacia, alfalfa, aneth, apple, apricot, artichoke, arugula, asparagus, avocado, banana, barley, beans, beet, blackberry, blueberry, broccoli, brussels sprouts, cabbage, canola, cantaloupe, carrot, cassaya, cauliflower, celery, cherry, cilantro, citrus, clementine, coffee, corn, cotton, cucumber, Douglas fir, eggplant, endive, escarole, eucalyptus, fennel, figs, forest trees, gourd, grape, grapefruit, honey dew, jicama, kiwifruit, lettuce, leeks, lemon, lime, loblolly pine, mango, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, tangerine, tea, tobacco, tomato, turf, a vine, watermelon, wheat, yams, and zucchini. Crop plants are defined as plants that are cultivated to produce one or more commercial product. Examples of such crops or crop plants include, but are not limited to, soybean, canola, rape, cotton (cottonseeds), sunflower, and grains such as corn, wheat, rice, and rye. The terms rape, rapeseed, and canola are used synonymously in the present disclosure.

The following examples are provided to better elucidate the practice of the present invention and should not be interpreted in any way to limit the scope of the present invention. Those skilled in the art will recognize that various modifications, additions, substitutions, truncations, etc., can be made to the methods and genes described herein while not departing from the spirit and scope of the present invention.

### EXAMPLES

### Example 1

### Stock Plant Material and Growth Conditions

Maize seeds were obtained from Monsanto branded seeds (Monsanto Company, St. Louis, Mo.) or Holdens Seeds Co. (Williamsburg, Iowa). The seeds were sown into 2.5 or 3.5 inch peat pots prepared with Metromix 200. Seeds and plants were grown under conditions of 16 hours light/8 hours dark at 22° C. to 23° C. (72° F.), in approximately 70% humidity. Green house or growth chamber lighting was adjusted to maintain light intensity between 650-850 micro Einstein/m² light intensity during wintertime and 300-500 micro Einstein/m² light intensity during summertime. Seedlings were transferred to 10 inch pots at V3-V4 stage. Seedlings or plants were watered daily, and fertilized three times a week from below with 200-ppm nitrogen using Peters 20-10-20 fertilizer. Micronutrients were added twice a week in the form of an iron mix (ferric ammonium citrate, 1500 g/5 gal and 1 quart Micrel Total/5 gal. Micrel Total was made by Growth Products Ltd., White Plains, N.Y.). Individual plants were hand-pollinated and ears were harvested at 40 days after pollination. Ears were dried for a minimum of four days at 37° C. and then hand-shelled.

Peters fertilizer, peat pots, iron mix, and all other supplies for growing corn seeds and seedlings were obtained from Hummert's International (Earth City, Mo.).

### Example 2

### Identification of Homologs, Paralogs or Orthologs

This example describes isolation of coding regions of the gene in accordance with the present invention. Homologs of the polynucleotides of the invention were identified from a cDNA library of the desired plant species.

For construction of cDNA libraries from plants, plant tissues were harvested and immediately frozen in liquid nitrogen and stored at −80° C. until total RNA extraction. Trizol reagent from Life Technologies (Gibco BRL, Life Technologies, Gaithersburg, Md.) was used for isolation of total RNA from different plant tissues as per the recommendation of the manufacturer. Poly A+ RNA (mRNA) was purified by using magnetic oligo dT beads essentially as recommended by the manufacturer (Dynabeads, Dynal Corporation, Lake Success, N.Y.).

The Superscript™ Plasmid System for cDNA synthesis and Plasmid Cloning (Gibco BRL, Life Technologies) were used for construction of cDNA libraries, following the conditions suggested by the manufacturer.

The cDNA libraries were plated on LB agar containing the appropriate antibiotics for selection and incubated at 37° C. for sufficient time to allow the growth of individual colonies. Single colonies from selective media were individually placed in each well of a 96-well microtiter plate containing LB liquid including the selective antibiotics. The plates were incubated overnight at approximately 37° C. with gentle shaking to promote growth of the cultures.

The plasmid DNA was isolated from each clone using QIAprep plasmid isolation kits, using the conditions recommended by the manufacturer (Qiagen Inc., Santa Clara, Calif.).

The template plasmid DNA clones were used for subsequent sequencing. For sequencing the cDNA libraries, a commercially-available sequencing kit, such as the ABI PRISM dRhodamine Terminator Cycle Sequencing Ready Reaction Kit with AmpliTaq® DNA Polymerase, FS, was used under the conditions recommended by the manufacturer (Perkin-Elmer Corp., Applied Biosystems Div., Foster City, Calif.). Sequencing was initiated from the 5′ end or 3′ end of each cDNA clone that generated the cDNA sequences disclosed herein. Entire inserts or only part of the inserts (expressed sequenced tags or ESTs) were sequenced. For sequencing, we used the 377 and 3700 DNA Sequencers with reagents provided by the vendor (Perkin-Elmer Corp., Applied Biosystems Div.).

The full-length and EST DNA sequences were used to search for homologs in various DNA sequence databases including GenBank. The combined dataset was then clustered and assembled using Pangea Systems (DoubleTwist, Oakland, Calif.) software identified as CAT v.3.2. First, the EST sequences were screened and filtered, e.g. high frequency words were masked to prevent spurious clustering; sequence common to known contaminants such as cloning bacteria were masked; high frequency repeated sequences and simple sequences were masked; unmasked sequences of less than 100 base pairs were eliminated. The thus-screened and filtered ESTs were combined and subjected to a word-based clustering algorithm that calculates sequence pair distances based on word frequencies and uses a single linkage method to group like sequences into clusters of more than one sequence, as appropriate. Clustered sequences were assembled individually using an iterative method based on PHRAP/CRAW/MAP, providing one or more self-consistent consensus sequences and inconsistent singleton sequences.

The above-described databases containing nucleotide and peptide sequences were queried with sequences of the present invention to obtain the homologues, orthologs or paralogs shown in Table 1.

Homologous proteins were identified using similarity searches: BLAST searches of the protein query sequences from the present invention were used to search the National Center for Biotechnology Information (NCBI) non-redundant amino acid database and Monsanto clustered EST data. The PFAM “globin” model was also used to search Monsanto clustered EST data using the HMMSEARCH program from the HMMER package (v.2.3.1, Sean Eddy, distributed by the author, Washington University, St. Louis). The open n reading frame in each recombinant polynucleotide sequence was identified by a combination of predictive and homology-based methods. The collections of sequences found were aligned using the HMMALIGN program from the HMMER package (v2.3.1, Sean Eddy, distributed by the author, Washington University, St. Louis), followed by manual editing of the alignment.

Phylogenetic analysis was then done to determine the evolutionary relationships between genes. From these relationships, functional similarity can be inferred. Phylogenetic analysis was done using programs in the PHYLIP (Phylogeny Inference Package) package version 3.6, distributed by the author (Felsenstein, J. 1993, Department of Genetics, University of Washington, Seattle).

“Percent sequence identity” is determined by comparing two optimally aligned sequences over a comparison window, wherein the portion of the polynucleotide or amino acid sequence in the comparison window may comprise additions or deletions for optimal alignment of the two sequences. Percent identity was calculated using the GCG GAP program version 10.3 RDB-Unix, provided by Accelrys Inc. (9685 Scranton Road, San Diego, Calif.). Comparisons were done using the coding sequence (CDS) region of all genes.

This example illustrates genes envisioned for use in accordance with aspects of the invention.

TABLE 1

| Nucleotide | Peptide | | |
|---|---|---|---|
| SEQ ID | SEQ ID | | Expression |
| NOs | NOs | Gene Annotation | Vector NOs |
| 1 | 39 | LIB3061-001-H7_FLI | PMON68857 |
| 2 | 40 | Synechocystis biliverdin | PMON67805 |
| | | reductase | |
| 3 | 41 | 700331819_FLI-corn | PMON68608 |
| | | FPPS 2 | |
| 4 | 42 | Receiver domain (TOC1- | PMON67811 |
| | | like) 3 | |
| 5 | 43 | 14-3-3-like protein D | PMON67817 |
| 6 | 44 | Synechocystis fructose- | PMON75488 |
| | | 1,6-bisphosphatase F-II | |
| 7 | 45 | PHE0000231 nucellin- | PMON72498 |
| | | like protein | |
| 8 | 46 | cytochrome P450 | PMON69470 |
| | | DWARF3 | |
| 9 | 47 | SKI4-like protein | PMON68885 |
| 10 | 48 | maize catalase-3 | PMON68400 |
| 11 | 49 | soy putative 2-cys | PMON74411 |
| | | peroxiredoxin | |
| 12 | 50 | HSP21-like protein | PMON73160 |
| 13 | 51 | soy HMG CoA synthase | PMON69476 |
| 14 | 52 | soy ttg1-like2 | PMON74408 |
| 15 | 53 | Synechocystis | PMON68401 |
| | | hypothetical sugar kinase- | |
| | | BAA10827 | |
| 16 | 54 | corn JMT-like protein 1 | PMON69498 |
| 17 | 55 | yeast GLC8-P41818 | PMON75473 |
| 18 | 56 | Synechocystis ssr2315- | PMON75484 |
| | | BAA17190 | |
| 19 | 57 | soy MKP 1 | PMON69492 |
| 20 | 58 | yeast YOR161c-Z75069 | PMON75489 |
| 21 | 59 | wheat nicotianamine | PMON69482 |
| | | aminotransferase | |
| 22 | 60 | corn G beta 2 | PMON74447 |
| 23 | 61 | yeast YLR179C- | PMON74431 |
| | | AAB67472 | |
| 24 | 62 | rice FPF1-like 1 | PMON68620 |
| 25 | 63 | rice FPF1-like 3 | PMON75303 |
| 26 | 64 | maize 60S acidic | PMON69485 |
| | | ribosomal protein P0 | |
| 27 | 65 | corn nucleotide-binding | PMON68623 |
| | | protein | |
| 28 | 66 | MADS affecting flowering | PMON68611 |
| | | 1-AAK37527 | |
| 29 | 67 | soy TGACG-motif- | PMON68648 |
| | | binding protein STF2 | |
| 30 | 68 | PHE0000647 corn | PMON74437 |
| | | unknown protein | |
| 31 | 69 | rice phytosulphokine | PMON75317 |
| | | SH27A-3201971 | |
| 32 | 70 | YPD1-Z74283 | PMON76311 |
| 33 | 71 | soy duf6 2 | PMON77854 |
| 34 | 72 | yeast hnRNP | PMON73830 |
| | | methyltransferase- | |
| | | CAA53689 | |
| 35 | 73 | corn F-box 125 [TIR1] | PMON78201 |
| 36 | 74 | yeast YDR209c-S61572 | PMON73827 |
| 37 | 75 | corn GS1-like protein | PMON73823 |
| 38 | 76 | corn sterol-C5(6)- | PMON75536 |
| | | desaturase 1 | |

### Example 3

This example describes construction of a plant expression vector used for transforming plants in accordance with the present invention. A representative DNA construct that can be used to transform a plant to express any protein of the invention is shown in FIG. 1.

A suitable plant transformation vector can comprise DNA constructs that are a combination of other DNA segments. These DNA segments provide replication function and antibiotic selection in the bacterial cells. For example, replication function can be provided by an E. coli origin of replication such as ori322 or a broad host range origin of replication such as oriV or oriRi. Antibiotic selection can be provided by a coding region for a selectable marker such as Spc/Str that encodes for Tn7 aminoglycoside adenyltransferase (aadA), conferring resistance to spectinomycin or streptomycin, or a gentamicin (Gm, Gent) selectable marker gene. One or more suitable promoters may be used to drive the expression of the selectable marker and the gene of interest. Any promoter that will work in a plant cell can be used, for example the rice actin promoter. Intronic sequences may also be inserted between the gene of interest and the promoter to improve the efficiency of expression in plants as shown in this figure. Plant transforming vectors may be designed with polylinker regions at appropriate locations with multiple restriction endonuclease sites. These sites may be used to provide a cloning site to clone genes in accordance with the present invention or to alter the expression cassette by changing different components of the cassette. Examples of such cloning sites include BglII, NcoI, EcoRI, SalI, Not1, XhoI, and other sites known to those skilled in the art of molecular biology. In the vector in FIG. 1, the gene of interest is followed by a termination region toward its 3′ end to stop translation of the gene. In addition to the above elements, the construct may also include an epitope tag, for example a Flag® peptide (catalog number F-3290, SIGMA, St. Louis, Mo.), at the 3′ termination region of gene of interest. The GATEWAY™ cloning technology (Invitrogen Life Technologies, Carlsbad, Calif.) was also used for construction of the vector of the invention shown in FIG. 1. GATEWAY™ technology uses phage lambda base site-specific recombination for vector construction, instead of restriction endonucleases and ligases. Assembly of DNA constructs were done by standard molecular biology techniques as described in Sambrook et al., “Molecular Cloning: A Laboratory Manual.”

For plant transformation, Agrobacterium tumefaciens ABI or LBA4404 was used as the host strain.

### Example 4

This example describes transformation of a plant with DNA constructs of the present invention.

Transgenic corn was produced by particle bombardment transformation methods as described in U.S. Pat. No. 5,424,412. The vector was digested with suitable restriction endonucleases to isolate a plant expression cassette that expresses the polypeptides disclosed herein in the plant. The desired expression cassette was purified by agarose gel electrophoresis and then bombarded into embryogenic corn tissue culture cells using a Biolistic® (Dupont, Wilmington, Del.) particle gun with purified isolated DNA fragments. Transformed cells were selected on selection media, such as glyphosate (N-phosphonomethyl glycine and its salts)-containing media, and whole plants were regenerated and grown under greenhouse conditions. Fertile seeds were collected, planted, and selected for the selectable marker by an appropriate screen. For example, if the selectable marker was the CP4 gene, glyphosate-resistant plants were selected. Selected plants were further subjected to cold vigor screening as described in Examples 5 and 6. Plants that were positive in both the cold vigor screen and the selectable marker screen were backcrossed into commercially acceptable corn germplasm by methods known in the art of corn breeding to produce commercial lines (Sprague et al., Corn and Corn Improvement 3^{rd} Edition, Am. Soc. Agron. Publ (1988)).

In some cases, transgenic corn plants were also produced by an Agrobacterium-mediated transformation method. A disarmed Agrobacterium strain C58 (ABI) harboring a desired DNA construct was used for the experiments. The desired construct was transferred into Agrobacterium by a triparental mating method (Ditta et al., Proc. Natl. Acad. Sci. 77:7347-7351). Liquid cultures of Agrobacterium were initiated from glycerol stocks or from a freshly streaked plate and grown overnight at a temperature ranging from 26° C. to 28° C. with shaking (approximately 150 rpm) to mid-log growth phase in liquid LB medium, pH 7.0 containing 50 mg/l kanamycin, 50 mg/l streptomycin and spectinomycin and 25 mg/l chloramphenicol with 200 μM acetosyringone (AS). The Agrobacterium cells were resuspended in the inoculation medium (liquid CM4C) and the density was adjusted to an OD₆₆₀ of 1. Freshly-isolated Type II immature HiII×LH198 and Hi II corn embryos were inoculated with Agrobacterium containing the desired DNA construct and co-cultured for 2 to 3 days in the dark at 23° C. The embryos were then transferred to delay media (N6 1-100-12/micro/Carb 500/20 μM AgNO3) and incubated at 28° C. for 4 to 5 days. All subsequent cultures were kept at this temperature. Coleoptiles were removed one week after inoculation. The embryos were transferred to the first selection medium (N61-0-12/Carb 500/0.5 mM glyphosate). Two weeks later, surviving tissues were transferred to the second selection medium (N61-0-12/Carb 500/1.0 mM glyphosate). Surviving callus was sub-cultured every 2 weeks until events could be identified. Usually this took three subcultures on 1.0 mM glyphosate. Once events were identified, tissues were bulked up for regeneration. For regeneration, callus tissues were transferred to the regeneration medium (MSOD, 0.1 μM ABA) and incubated for two weeks. The regenerating calli were transferred to a high sucrose medium and incubated for two weeks. The plantlets were transferred to MSOD media in culture vessel and kept for two weeks. Then the plants with roots were transferred into soil.

Soybean plants are transformed using an Agrobacterium-mediated transformation method, as described by Martinell (U.S. Pat. No. 6,384,301, herein incorporated by reference). For this method, overnight cultures of Agrobacterium tumefaciens containing the plasmid that includes a gene of interest are grown to log phase and then diluted to a final optical density at 660 nm (OD₂₆₀) ranging from 0.3 to 0.6 using standard methods known to one skilled in the art. These cultures are used to inoculate the soybean embryo explants prepared as described below.

Commercially available soybean seeds (e.g., Asgrow A3244) are germinated overnight and the meristematic tissue is excised. The excised tissue is placed in a wounding vessel and mixed with the Agrobacterium culture described above. The entire tissue is wounded using sonication. Following the wounding, explants are placed in co-culture for 2-5 days, at which point they are transferred to selection media, i.e., WPM (as described on page 19 of U.S. Pat. No. 6,211,430, incorporated herein by reference) with 75 mM glyphosate (plus antibiotics to control Agrobacterium overgrowth), for 6-8 weeks to allow selection and growth of transgenic shoots. Phenotype-positive shoots are harvested approximately 6-8 weeks post transformation and placed into selective rooting media (BRM, as described in Table 3 of U.S. Pat. No. 6,384,301) with 25 mM glyphosate for 3-5 weeks. Shoots producing roots are transferred to the greenhouse and potted in soil. Shoots that remain healthy on selection, but do not produce roots are transferred to non-selective rooting media (BRM without glyphosate) for up to two weeks. Roots from the shoots that produced roots off selection are tested for expression of the plant selectable marker before they are transferred to the greenhouse and potted in soil. Plants are maintained under standard greenhouse conditions until seed harvest (R1).

### Example 5

This example describes a cold germination assay for transgenic corn seeds of the present invention that was used for testing of expected performance of seed under desired conditions. The cold germination assay was designed to measure the “Germination Index” of seeds under cold conditions as indicative of seedling vigor under stressed conditions.

Two sets of seeds were used for the experiment. The first set consisted of different positive transgenic events where the genes of the present disclosure were expressed in the seed. The second seed set consisted of wild-type lines of corn that were grown in the same nursery where the transgenic events were grown. All seeds were treated with the fungicide “Captan.” 0.43 mL Captan was applied per 45 g of corn seed by mixing it well and drying the fungicide prior to the experiment.

For every event, ten transgenic corn kernels were placed embryo side down on blotter paper within an individual cell (8.9×8.9 cm) of a germination tray (54×36 cm). For every event there were five replications (five trays). Trays were placed at 9.7° C. for 24 days in the dark. Germination counts take place on the 10th, 11th, 12th, 13th, 14th, 17th, 19th, 21st, and 24th day after the start date of the experiment. Seeds were considered germinated if the emerged radicle size was one cm.

The germination index was calculated as per:
Germination index=(Σ([T+1−nᵢ]*[Pᵢ−Pᵢ₋₁]))/T,
where T was the total number of days for which the germination experiment was performed. The number of days after planting was defined by n. “i” indicated the number of times the germination had been counted, including the current day. P was the percentage of seeds germinated during any given rating. Statistical differences were calculated between the transgenic events and the wild type control.

After statistical analysis, the positive events that show a statistical significance at a p level of less than 0.1 relative to wild-type controls advance to a secondary cold screen. The secondary cold screen was conducted in the same manner as the primary screen, but the number of repetitions was increased to ten. Statistical analysis of the data from the secondary screen was conducted to identify the positive events that show a statistical significance at a p level of less than 0.05 relative to wild type controls.

The results of this example are compiled in Table 2, which shows increased cold vigor for seeds that harbor selected transgenes of the invention as compared to non-transgenic seeds when cold vigor is measured in terms of the “Germination Index.” X indicates that the tested transgenic seeds performed better than the control seeds in a statistically significant manner in both the initial screen and the confirmation screen (p<0.1 for initial screen and p<0.05 for confirmation screen).

TABLE 2

| Nuc. | Pep. | | |
|---|---|---|---|
| SEQ | SEQ | | Germination |
| NO. | NO. | Gene Annotation | index |
| 1 | 39 | LIB3061-001-H7_FLI | X |
| 2 | 40 | Synechocystis biliverdin reductase | X |
| 4 | 42 | Receiver domain (TOC1-like) 3 | X |
| 5 | 43 | 14-3-3-like protein D | X |
| 7 | 45 | PHE0000231 nucellin-like protein | X |
| 8 | 46 | cytochrome P450 DWARF3 | X |
| 10 | 48 | maize catalase-3 | X |
| 11 | 49 | soy putative 2-cys peroxiredoxin | X |
| 12 | 50 | HSP21-like protein | X |
| 16 | 54 | corn JMT-like protein 1 | X |
| 17 | 55 | yeast GLC8-P41818 | X |
| 19 | 57 | soy MKP 1 | X |
| 20 | 58 | yeast YOR161c-Z75069 | X |
| 21 | 59 | wheat nicotianamine aminotransferase | X |
| 24 | 62 | rice FPF1-like 1 | X |
| 26 | 64 | maize 60S acidic ribosomal protein P0 | X |
| 27 | 65 | corn nucleotide-binding protein | X |
| 28 | 66 | MADS affecting flowering 1-AAK37527 | X |
| 29 | 67 | soy TGACG-motif-binding protein STF2 | X |
| 32 | 70 | YPD1-Z74283 | X |

### Example 6

This example describes the early seedling growth assay for transgenic corn seeds of the present invention. The early seedling growth assay was designed to measure the seedling vigor produced by selected seeds of the invention in desired conditions.

Two sets of seeds were used for the experiment. The first set consisted of different positive transgenic events where the genes of the present invention were expressed in the seed. The second seed set consisted of wild type lines of corn that were grown in the same nursery where the transgenic events were grown. All seeds were treated with the fungicide “Captan.” 0.43 mL Captan was applied per 45 g of corn seeds by mixing it well and drying the fungicide prior to the experiment.

Seeds were grown in germination paper for the early seedling growth assay. Three 12″ by 18″ pieces of germination paper (Anchor Paper #SD7606) were used for each entry in the test. The papers were wetted in a solution of 0.5% KNO₃ and 0.1% Thyram.

Fifteen seeds were placed in a line on each paper, evenly spaced down the length of the paper. The fifteen seeds were positioned on the paper such that the radical would grow downward, the longer distance to the paper's edge. The wet paper was rolled up starting from one of the short ends. The paper was rolled evenly and tightly enough to hold the seeds in place. The roll was secured into place with two large paper clips, one at the top and one at the bottom. The rolls were incubated in a growth chamber at 23° C. for three days in a randomized complete block design within an appropriate container. The chamber was set for 65% humidity with no light cycle. For the cold stress treatment, the rolls were then incubated in a growth chamber at 12° C. for 12 days. The chamber was set for 65% humidity with no light cycle.

After the appropriate treatment, the germination papers were unrolled and the seeds that did not germinate were discarded. The length of the radicle (primary root), the coleoptile (primary shoot), and the total seedling were measured for each seed and the data were recorded.

Raw data were statistically analyzed for each event. After statistical analysis, the events that show a statistical significance at a p level of less than 0.1 relative to wild-type controls were advanced to a secondary cold screen. The secondary cold screen was conducted in the same manner as the primary screen, but the number of repetitions was increased to five. Statistical analysis of the data from the secondary screen was conducted to identify the events that show a statistical significance at p level of less than 0.05 relative to wild-type controls.

The results of this example are compiled in Table 3, which shows increased cold vigor for seeds that harbor selected transgenes of the invention as compared to non-transgenic seeds when cold vigor is measured by performing the “early seedling growth assay.” Results of the early seedling growth assay for each transgene are presented in terms of root length, shoot length, and their combinations. X indicates that the tested transgenic seeds performed better than the control seeds in a statistically significant manner in both the initial screen and the confirmation screen (p<0.1 for initial screen and p<0.05 for confirmation screen).

TABLE 3

| | | | Root, | | | |
|---|---|---|---|---|---|---|
| | | | Shoot | | | |
| | Pep. | | and | Root and | | |
| Nuc. | SEQ | | Total | Total | Shoot | Root |
| SEQ NO. | NO. | Gene Annotation | Lenghts | Lengths | Length | Length |
| 3 | 41 | 700331819_FLI-corn | X | | | |
| | | FPPS 2 | | | | |
| 6 | 44 | Synechocystis fructose- | X | | | |
| | | 1,6-bisphosphatase F-II | | | | |
| 9 | 47 | SKI4-like protein | | X | | |
| 13 | 51 | soy HMG CoA synthase | | X | | |
| 14 | 52 | soy ttg 1-like 2 | | | | X |
| 15 | 53 | Synechocystis hypothetical | | | | X |
| | | sugar kinase - BAA10827 | | | | |
| 18 | 56 | Synechocystis ssr2315 - | | | X | |
| | | BAA17190 | | | | |
| 22 | 60 | corn G beta 2 | | X | | |
| 23 | 61 | yeast YLR179C- | | | | X |
| | | AAB67472 | | | | |
| 25 | 63 | rice FPF1-like 3 | X | | | |
| 30 | 68 | PHE0000647 corn | | | | X |
| | | unknown protein | | | | |
| 31 | 69 | rice phytosulphokine | X | | | |
| | | SH27A - 3201971 | | | | |
| 33 | 71 | soy duf6 2 | | X | | |
| 35 | 73 | corn F-box 125 [TIR1] | | | | X |
| 36 | 74 | yeast YDR209c-S61572 | X | | | |
| 37 | 75 | corn GS1-like protein | | X | | |
| 38 | 76 | corn sterol-C5(6)- | | X | | |
| | | desaturase 1 | | | | |

We claim:

1. A transgenic seed comprising a DNA construct capable of expressing a functional polypeptide selected from the group consisting of SEQ ID NO: 39 to SEQ ID NO: 76 or from the group consisting of polypeptide sequences substantially homologous to SEQ ID NO: 39 to SEQ ID NO: 76 at least during the period of seed germination and early seedling growth.

2. A transgenic seed comprising a DNA construct capable of expressing a functional polypeptide selected from the group consisting of SEQ ID NO: 39 to SEQ ID NO: 76 when germinated in a field under sub-optimal growth conditions, providing a seedling with enhanced cold tolerance.

3. A transgenic corn seed comprising a DNA construct capable of expressing a functional polypeptide with at least 75% identity to a polypeptide selected from a group consisting of SEQ ID NO: 39 to SEQ ID NO: 76, characterized by a germination index value ranging from 48 to 150 at a temperature ranging from about 9.0° C. to 9.8° C. and having a percent germination of seed greater than 80% at a temperature ranging from about 8.0° C. to 9.3° C.

4. A plant cell with a stably integrated DNA construct comprising a promoter that is functional in plant cells at least during the period of seed germination and early seedling growth and that is operably linked to a polynucleotide sequence that encodes a functional protein, wherein said polynucleotide sequence is selected from the group consisting of SEQ ID NO: 1 through SEQ ID NO: 38 or from the group consisting of polynucleotide sequences substantially homologous to SEQ ID NO: 1 through SEQ ID NO: 38; wherein said plant cell is selected from a population of plant cells with said recombinant DNA by screening seeds or plants that are regenerated from plant cells in said population for enhanced cold tolerance as compared to control plants or seeds of the same species that do not contain said recombinant DNA.

5. The plant cell of claim 4, wherein the plant cell is selected from the group consisting of corn, soybean, wheat, cotton, rice, rapeseed, and alfalfa.

6. A transgenic plant comprising a plurality of the plant cell of claim 5.

7. A transgenic seed comprising a plurality of the plant cell of claim 5.

8. A method of producing a transgenic seed with enhanced cold tolerance, comprising the steps of:
a) transforming a plant cell with a DNA construct comprising a promoter that is functional in plant cells at least during the period of seed germination and early seedling growth and that is operably linked to a polynucleotide sequence that encodes a functional protein, wherein said polynucleotide sequence is selected from the group consisting of SEQ ID NO: 1 through SEQ ID NO: 38 or from the group consisting of polynucleotide sequences substantially homologous to SEQ ID NO: 1 through SEQ ID NO: 38;
b) regenerating said transformed plant cell into a transgenic plant comprising said DNA construct;
c) collecting a population of transgenic seeds from said transgenic plant;
d) screening said population of transgenic seeds for enhanced cold tolerance as compared to control seeds of the same species that do not contain said DNA construct; and
e) selecting from said population one or more transgenic seeds with enhanced cold tolerance.

9. A transgenic seed produced by the method of claim 8.

10. The method of claim 8, wherein said plant cell is selected from the group consisting of corn, soybean, wheat, cotton, rice, rapeseed, and alfalfa.

11. A transgenic plant produced by planting the seed of claim 1, 2, 3, 7, or 9.

12. The transgenic seed of claim 1, 2, 3, 7, or 9, wherein the transgenic seed is coated with a seed coating permitting imbibition and germination at low soil temperature.

13. The transgenic seed of claim 12, wherein said seed coating comprises an agent selected from the group consisting of a fungicide seed coating, a bactericide seed coating, an insecticide seed coating, a plant hormone seed coating, a nutrient seed coating, a microbial inoculum seed coating, a color seed coating, an avian repellent seed coating and a rodent repellent seed coating.

14. The transgenic seed of claim 1, 2, 3, 7, or 9, wherein the seed has enhanced cold vigor demonstrable by a cold germination assay showing an average temperature of germination at least about two degrees Celsius less than the average temperature of germination of a non-transgenic seedling of a comparable variety.

15. The transgenic seed of claim 12, wherein the seed has enhanced cold vigor demonstrable by a cold germination assay showing an average temperature of germination at least about two degrees Celsius less than the average temperature of germination of a non-transgenic seedling of a comparable variety.

16. A hybrid seed, wherein the transgenic seed of claim 1, 2, 3, 7, 9, 12, or 13 is grown into a plant to the reproductive stage and is crossed with a second plant to produce hybrid seed.

17. The hybrid seed of claim 16, wherein said second plant is resistant to an herbicide selected from a group consisting of a glyphosate herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides, glufosinate herbicides and auxin-like herbicides.

18. A method of producing a crop, said method comprising:
planting the transgenic seed of claim 1, 2, 3, 7, 9, 12, 13, 16, or 17; and
harvesting a resulting crop.

19. The method of claim 18, wherein said crop is a terminal crop.

20. The method of claim 18, whereby yield of the crop from the plant produced by the transgenic seed is increased as compared to yield of the crop from a plant produced by non-transgenic seed of similar genotype.

21. The method of claim 18, whereby root biomass of the plant seedling produced by the transgenic seed is increased as compared to the root biomass of a plant seedling produced by non-transgenic seed of similar genotype.

22. The method of claim 18, whereby shoot biomass of the plant seedling produced by the transgenic seed is increased as compared to the shoot biomass of a plant seedling produced by non-transgenic seed of similar genotype.

23. A method of extending the cool weather-growing season of a crop plant, said method comprising:
planting the transgenic seed of claim 1, 2, 3, 7, 9, 12, 13, 16, or 17, under conditions including time of planting effective to extend the cool weather-growing season.

24. The method of claim 23, further comprising:
planting said transgenic seed at least one week earlier than the planting of a non-transgenic seed of similar genotype.

25. A crop produced by the method of claim 18 or 23.
